(19) United States
(12) Patent Application Publication
(10) Pub. No.: US 2026/0345347 A1
(43) Pub. Date: Nov. 5, 2026

(54) Spinal Implants And Implant Inserters

(71) Applicant: VB Spine LLC, Leesburg, VA (US)

(72) Inventors: Bryan D. Milz, Milford, NJ (US); Jordan Holder, Chester, NY (US); Michael Curtis, Kildare (IE)

(21) Appl. No.: 19/663,339

(22) Filed: Apr. 30, 2026

Related U.S. Application Data
(60) Provisional application No. 63/797,471, filed on Apr. 30, 2025.

(51) Int. Cl. A61F 2/46; A61F 2/44

(52) U.S. Cl. CPC A61F 2/4611; A61F 2/4455; A61F 2002/4622; A61F 2002/4625

(57) ABSTRACT

An inserter for a spinal implant may include inner shaft having a longitudinal slit at a distal end defining a first arm and a second arm. Each arm may include a concave portion on an internal surface, and the concave portions may define a space. The inserter may also include an outer shaft surrounding a portion of the inner shaft. The outer shaft may include a grasping pin positioned within the space between the first and second arms. The grasping pin may have a first diameter larger than a first distance between the first and second arms at a proximal end of the space so that, when the grasping pin is positioned at the proximal end of the space, the first and second arms are an expanded position.

### CROSS-REFERENCE TO RELATED APPLICATION

This application claims the benefit of the filing date of United States Provisional Patent Application No. 63/797,471, filed Apr.30, 2025, the disclosure of which is hereby incorporated by reference herein in its entirety.

### BACKGROUND

Transforaminal lumbar interbody fusion (TLIF) spinal fusion procedures include inserting an implant, which are often curved, into a patient's disc space through an angled posterior trajectory. TLIF implants are commonly oriented in line with an implant inserter when first assembled. Once the TLIF implant has entered the disc space, the TLIF implant is rotated so that the implant ultimately aligns with an anterior aspect of the patient's vertebra.

The manner in which a TLIF implant is secured to an inserter can vary. Preferably, the connection must allow rotation of the implant within the disc space. For instance, certain existing inserters have a threaded mechanism that connects with the TLIF implant and a rotating mechanism either in the implant or inserter to facilitate rotation of the TLIF implant. This can include a rotating pin, a gear, a pulley, or levers. While some existing inserters and/or TLIF implants actively rotate the implant for a surgeon, other inserter/implant assemblies enable a surgeon to passively accommodate manual rotation.

Some existing inserters include discreet rotational locations that a surgeon and/or mechanism can rotate to, but surgeons are unable to regain control of the TLIF implant when rotating between rotational locations. Other existing inserters allow continuous rotation from one angle to another and allow locking of the implant at any point between the continuous rotation. However, these continuous inserters often require multiple hands to engage and disengage the rotation lock.

Accordingly, there is a need for inserters with improved control and ease of use.

### BRIEF SUMMARY

The present disclosure is generally directed to various inserters and various implants with features complementary to the various inserters. The inserters of the present disclosure provide improved control of articulation of an implant and improved ease of use for a more efficient procedure.

A first implant includes a post engageable by a first inserter. The design of both the first implant and first inserter enable the first inserter to be moved between an unlocked position, a semi-locked position, and a locked position. In the unlocked position, an implant can be engaged or disengaged without much resistance, and inner shafts are proximally translated so that the grasper assembly is expanded because of a pin in a longitudinal slit of the grasper assembly. In the semi-locked position, an implant can be rotated about an axis but is otherwise retained by the inserter, and the inner shafts are in a middle position so that the grasper assembly is in a neutral position because the pin is not in contact with the longitudinal slit of the grasper assembly. In the locked position, an implant cannot be disengaged or rotated, and the inner shafts are in a distally translated so that the grasper assembly is compressed because an outer shaft surrounds a portion of the grasper assembly in the locked position.

A second implant includes a recess having convex portions engageable by concave portions on an inserter. Like the first inserter and implant, the design of both the second implant and second inserter enable the second inserter to be moved between an unlocked position, a semi-locked position, and a locked position. In the unlocked position, an implant can be engaged or disengaged without much resistance, and a shaft is proximally translated so that the shaft does not compress the grasper assembling against the convex portions of the implant. In the semi-locked position, an implant can be rotated about an axis but is otherwise retained by the inserter, and the shaft is in a middle position so that the grasper assembly is in a neutral position. In the locked position, an implant cannot be disengaged or rotated, and the inner shafts are in a distally translated so that the grasper assembly is compressed because the shaft is fully positioned between the convex portions of the implant, thus compressing the arms against the convex portions.

A third implant includes a recess to receive an inserter attachment. The inserter attachment has a head including first and second portions that compress upon entry into the recess of the implant. Once fully received, the first and second portions relax from the compressed position so that inserter attachment is secured within recess. An inserter may then be connected to the secured inserter attachment.

In one aspect, an inserter for a spinal implant includes at least one inner shaft having a longitudinal slit at a distal end defining a first arm and a second arm. Each arm includes a concave portion on an internal surface, and the concave portions define a space. The inserter also includes an outer shaft surrounding a portion of the at least one inner shaft and having a grasping pin positioned within the space between the first and second arms. The grasping pin has a diameter larger than a first distance between the first and second arms at a proximal end of the space so that, when the grasping pin is positioned at the proximal end of the space, the first and second arms are in an expanded position.

In some examples, the diameter of the grasping pin is larger than a second distance between the first and second arms at a point between the proximal end and a distal end of the space so that, when the grasping pin is positioned at the point between the proximal and distal ends of the space, the first and second arms are in a neutral position.

In some examples, the outer shaft surrounds a portion of the first and second arms when the grasping pin is positioned at a distal end of the space so that the first and second arms are compressed.

In some examples, the space is a teardrop shape so that a distance between the first and second arms at the proximal end of the space is smaller than a distance between the first and second arms at a distal end of the space.

In some examples, the first arm and second arm each include a rounded end. The rounded ends face each other and define an engagement space configured to receive a portion of an implant.

In some examples, the inserter further comprises a lever pivotally connected to the inner shaft such that depression or elevation of the lever relative to the outer shaft translates the inner shaft in a proximal-distal direction.

In some examples, the inserter further comprises a lever lock slidable relative to the inner shaft.

In some examples, the lever further comprises a tab configured to be secured under the lever lock.

In another aspect, an inserter for a spinal implant includes a handle having an opening at a proximal end and at least one inner shaft translatable within the handle. The at least one inner shaft includes a grasper assembly at a distal end and a fin at a proximal portion. The inserter also includes an unlocking button through the opening of the handle and surrounding the at least one inner shaft. The unlocking button includes a barrier disposed within a hole through which the at least one inner shaft is received. The barrier abuts a distal end of the fin.

In some examples, a diameter of the hole is larger than the fin so that, when the unlocking button is compressed, the fin is received through the hole.

In some examples, the fin includes a ramped surface that tapers to a proximal end.

In some examples, the unlocking button comprises a top portion transverse to a side portion. The top portion includes a spring configured to control movement of the unlocking button along an axis when the unlocking button is compressed.

In yet another aspect, an inserter for a spinal implant includes a handle surrounding at least one inner shaft. The at least one inner shaft includes a grasper assembly on a distal end. The inserter also includes a lever pivotally connected to the at least one inner shaft such that movement of the lever translates the at least one inner shaft along an axis. The lever includes a tab at a proximal end. The inserter also includes a lever lock positioned on the handle and translatable along the axis such that translation of the lever lock in a distal direction secures the tab of the lever within a space between the lever lock and the handle.

In some examples, the lever is connected to the handle at a first connection point and to a fulcrum link at a second connection point. The fulcrum link is connected to the at least one inner shaft at a third connection point.

In some examples, the lever lock includes two protrusions positioned on either side of the at least one inner shaft to enable translation along the axis.

In some examples, the grasper assembly includes a first arm and a second arm. The first arm and second arm each include a rounded end. The rounded end face each other and define an engagement space configured to receive a portion of an implant.

In yet another aspect, a method of inserting a spinal implant includes translating at least one inner shaft along an axis in a distal direction so that a grasping pin connected to an outer shaft surrounding a portion of the at least one inner shaft is moved to a proximal end of a space within a grasper assembly. During this step, the grasper assembly is at a distal end of the at least one inner shaft. The grasping pin has a width larger than a first distance at the proximal end of the space and perpendicular to the axis such that a first arm and a second arm in the grasper assembly are moved to an expanded position. The method also includes engaging a post within a recess of an implant between a grasper assembly. The method also includes translating the at least one inner shaft in a proximal direction so that an outer shaft surrounds a portion of the at least one inner shaft and compresses the first and second arms.

In some examples, the method also includes rotating the engaged implant about the post.

In some examples, the first translating step further comprises moving a lever pivotally connected to the at least one inner shaft in an upward direction.

In some examples, the second translating step further comprises moving the lever in a downward direction.

In some examples, the method also includes translating a lever lock in a distal direction so that a tab on the lever is secured under a winged portion of the lever lock.

In some examples, the second translating step positions a proximal end of the implant against a distal end of the outer shaft. The end of the outer shaft is complementary in shape to the proximal end of the implant.

In yet another aspect, an implant system includes an implant body having a recess and an inserter attachment with a longitudinal slit defining a first portion and a second portion. The inserter attachment has a width larger than an opening of the recess so that the first and second portions compress upon entry in the recess.

In some examples, the inserter attachment is internally threaded.

In some examples, the inserter includes an inserter threadedly connected to the inserter attachment.

In some examples, the inserter attachment is spherical.

In yet another aspect, a system comprises spinal implant and an inserter for the spinal implant. The spinal implant comprises an inner shaft including a longitudinal slit at a distal end defining a first arm and a second arm. Each arm includes a concave portion on an internal surface. The concave portions define a space. The spinal implant also comprises an outer shaft surrounding a portion of the inner shaft and comprising a grasping pin positioned within the space between the first and second arms. The grasping pin has a diameter larger than a first distance between the first and second arms at a proximal end of the space so that, when the grasping pin is positioned at the proximal end of the space, the first and second arms are in an expanded position.

In yet another aspect, a system comprises spinal implant and an inserter for the spinal implant. The spinal implant comprises a handle including an opening at a proximal end. The spinal implant also comprises an inner shaft translatable within the handle. The inner shaft comprises a grasper assembly at a distal end and a fin at a proximal portion. The spinal implant comprises an unlocking button through the opening of the handle and surrounding the inner shaft. The unlocking button includes a barrier disposed within a hole through which the inner shaft is received. The barrier abuts a distal end of the fin.

### BRIEF DESCRIPTION OF THE DRAWINGS

FIG. 1A is a side perspective view of an implant, in accordance with an aspect of the present disclosure.

FIG. 1B is a side perspective view of the implant of FIG. 1A.

FIG. 1C is a top view of the implant of FIG. 1A, which is a mirror image of a bottom view.

FIG. 2A is a side perspective view of an inserter in an unlocked position, in accordance with an aspect of the present disclosure.

FIG. 2B is a side perspective view of the inserter of FIG. 2A in a semi-locked position.

FIG. 2C is a top and side perspective view of the inserter of FIG. 2A in a locked position.

FIG. 2D is an exploded view of the inserter of FIG. 2A.

FIG. 2E is an enlarged top view of a grasper assembly of the inserter of FIG. 2A in the unlocked position, with certain portions hidden or partially hidden.

FIG. 2F is an enlarged top view of a grasper assembly of the inserter of FIG. 2A in a semi-locked position, with certain portions hidden or partially hidden.

FIG. 2G is an enlarged top view of a grasper assembly of the inserter of FIG. 2A in a locked position, with certain portions hidden or partially hidden.

FIG. 2H is an enlarged top view of a portion of the inserter of FIG. 2A in the unlocked position, without certain portions hidden or partially hidden.

FIG. 2I is an enlarged top view of a grasper assembly of the inserter of FIG. 2A in a semi-locked position, without certain portions hidden or partially hidden.

FIG. 2J is an enlarged top view of a portion of the inserter of FIG. 2A in a locked position, without certain portions hidden or partially hidden.

FIG. 2K is a top view of a portion of the inserter of FIG. 2A, with certain portions hidden or partially hidden.

FIG. 2L is a back perspective view of an unlocking button of the inserter of FIG. 2A.

FIG. 2M is another top view of a portion of the inserter of FIG. 2A, with certain portions hidden or partially hidden.

FIG. 2N is a top view of a portion of the inserter of FIG. 2C, with certain portions hidden or partially hidden.

FIG. 3A is a top view of an implant, in accordance with another aspect of the present disclosure.

FIG. 3B is a perspective view of the implant of FIG. 3A.

FIG. 3C is a first side view of the implant of FIG. 3A

FIG. 3D is another perspective view of the implant of FIG. 3A.

FIG. 4A is a side view of an inserter in an unlocked position, in accordance with an aspect of the present disclosure.

FIG. 4B is a side view of the inserter of FIG. 4A in a semi-locked position and engaged with the implant of FIG. 3A, with a portion hidden.

FIG. 4C is a side view of the inserter of FIG. 4A in a locked position and engaged with implant 300, with a portion hidden.

FIG. 4D is a side view of a grasper assembly of the inserter of FIG. 4A, with certain portions hidden or partially hidden.

FIG. 4E is a top view of the grasper assembly of FIG. 4A, which is a mirror image of a bottom view, without certain portions hidden or partially hidden.

FIG. 4F is a perspective view of the grasper assembly of FIG. 4A .

FIG. 4G is a cross-sectional view of the inserter of FIG. 4A engaged with the implant of FIG. 3A, the cross-section taken along line A-A in FIGS. 3A and 4E.

FIG. 4H is a cross-sectional view of the grasper assembly of FIG. 4A in a semi-locked position engaged with the implant of FIG. 3A, the cross-section taken along line A-A in FIGS. 3A and 4E.

FIG. 4I is a cross-sectional view of the grasper assembly of FIG. 4A in a locked position engaged with the implant of FIG. 3A, the cross-section taken along line A-A in FIGS. 3A and 4E.

FIG. 5 is a perspective view of an implant and inserter, in accordance with another aspect of the present disclosure.

FIG. 6 is a perspective view of an implant and inserter, in accordance with another aspect of the present disclosure.

FIG. 7A is a top perspective view of an implant assembly, in accordance with another aspect of the present disclosure.

FIG. 7B is an exploded view of the implant assembly of FIG. 7A.

FIG. 7C is a top view of the implant of FIG. 7A.

FIG. 7D is a cross-sectional view of the implant of FIG. 7A taken along line B-B in FIG. 7C.

FIG. 7E is a side view of the implant of FIG. 7A.

### DETAILED DESCRIPTION

Particular aspects of the present disclosure will be described herein with reference to the accompanying drawings. As shown in the drawings and as described throughout the following description, the term "proximal" should be understood as referring to the portion of a structure that is closer to a clinician during use and the term "distal" should be understood as referring to the portion of a structure that is farther from the clinician during use. Also, as used herein, the terms “substantially,” “generally,” and “about” are intended to mean that slight deviations from absolute are included within the scope of the term so modified. Wherever possible, and for brevity, the same or like reference numbers will be used throughout the drawings to refer to the same or like features within a different series of numbers (e.g., 100-series, 200-series, etc.).

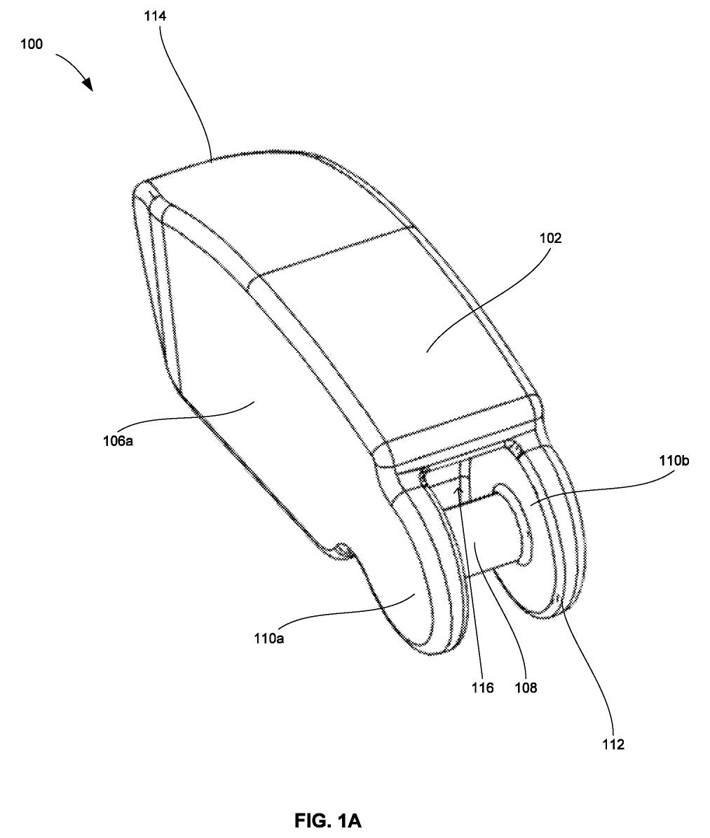

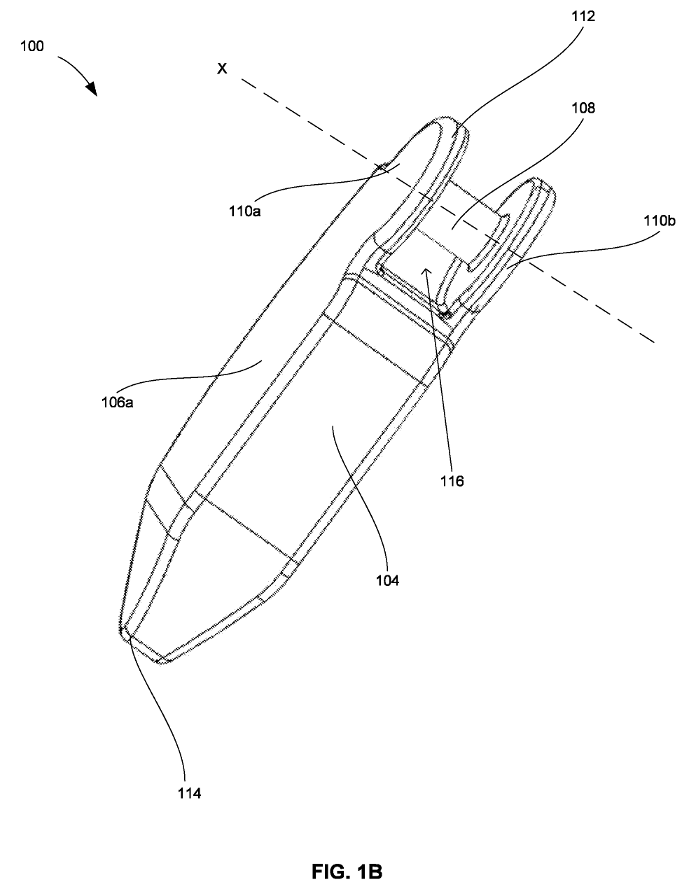

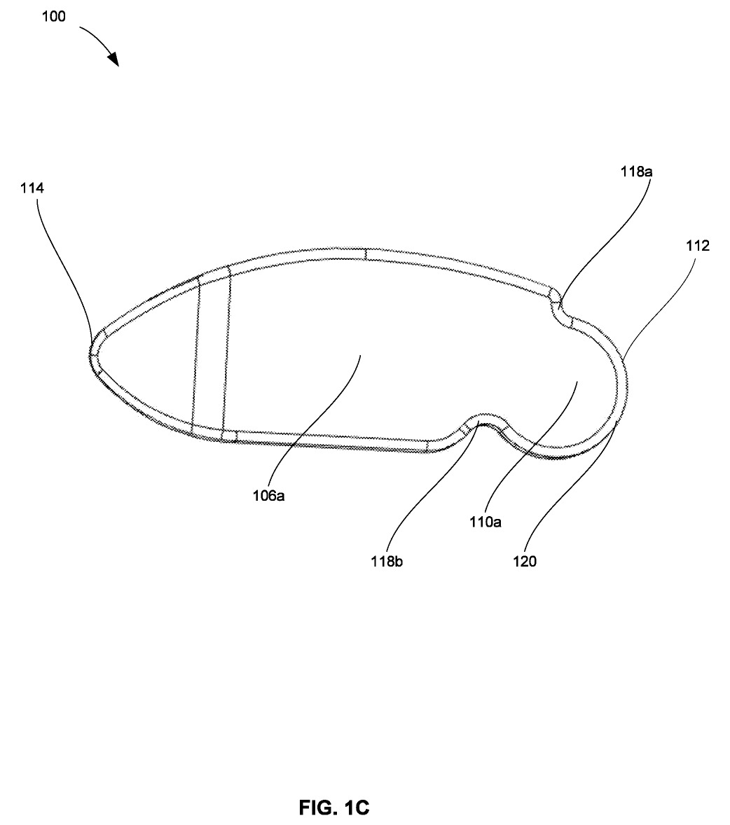

FIGS. 1A-1C depict an implant 100, according to an aspect of the present disclosure. Implant 100 includes a first lateral side 102, a second lateral side 104, a superior side 106a, an inferior side (not shown), a posterior side 112, and an anterior side 114. Implant 100 also includes at posterior side 112 a first extension 110a on superior side 106a and a second extension 110b on inferior side (not shown). First and second extensions 110a, 110b define a recess 116 therebetween and a post 108 within recess 116 and connected to first and second extensions 110a, 110b.

The outer configuration of implant 100 has a generally curved, kidney bean-like shape, and thus is consistent with the shapes of existing interbody implants used in TLIF techniques. However, the shape and size of implant 100 can vary depending upon a particular use. As best shown in FIG. 1C, first and second extensions 110a, 110b define indents in the overall implant shape. Specifically, first and second extensions 110a, 110b include a first concave portion 118a, which is connected to a convex portion 120, which is connected to a second concave portion 118b. Second concave portion 118b has a larger diameter than first concave portion 118b. The shape formed by first concave portion 118a, convex portion 120, and second concave portion 118b enables implant 100 to rotate while engaged in an inserter, such as inserter 200. In other aspects, implant 100 may have protrusions of any size and shape that can engage a bone.

Similarly, post 108 is intended to be received within the arms of an inserter, as is discussed below. Post 108 is shown as being cylindrical in shape but could be any shape suitable for grasping by corresponding portions of an inserter. Post 108 is static and positioned within recess 116 so that there is a gap between post 108 and the body of implant 100 such that an inserter has sufficient clearance to rotate implant 100 about an axis X parallel to and/or coincident with post 108.

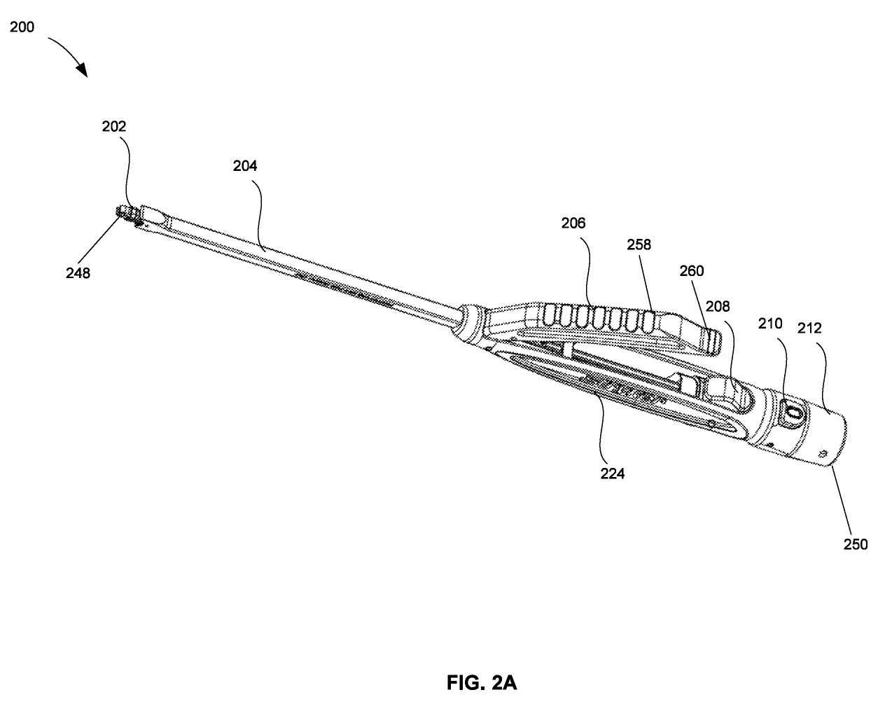

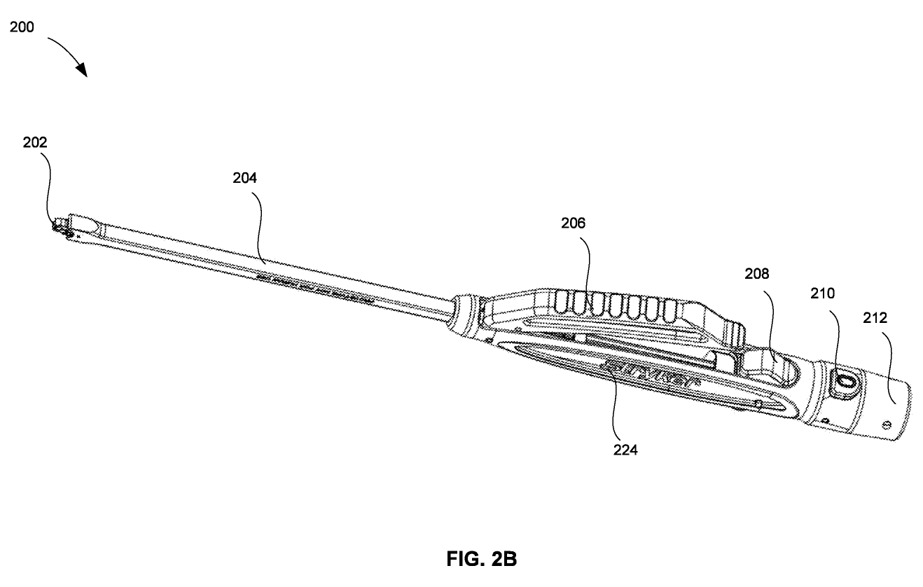

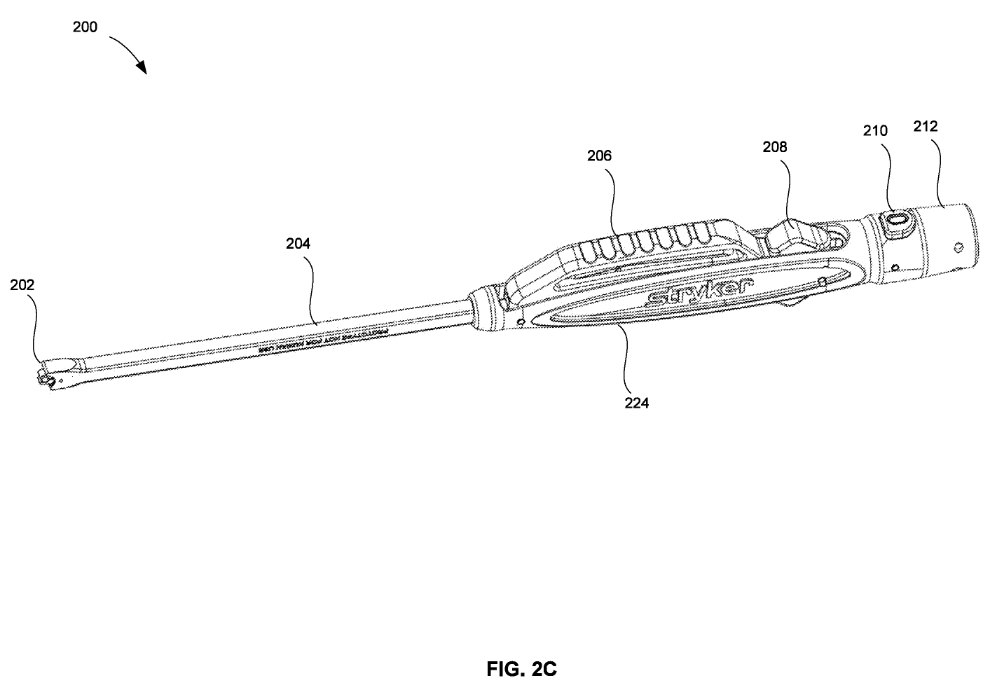

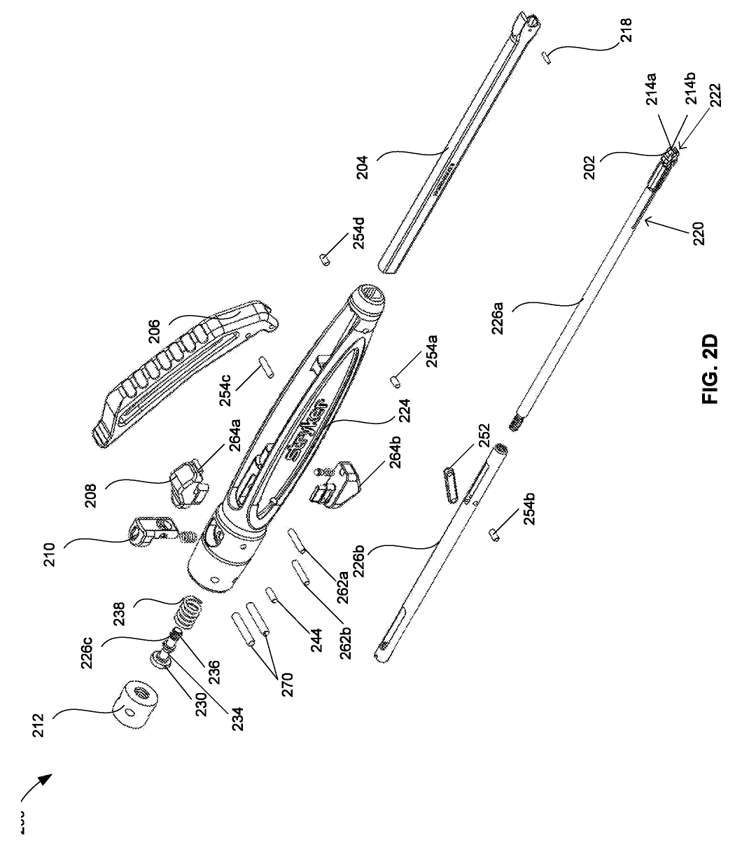

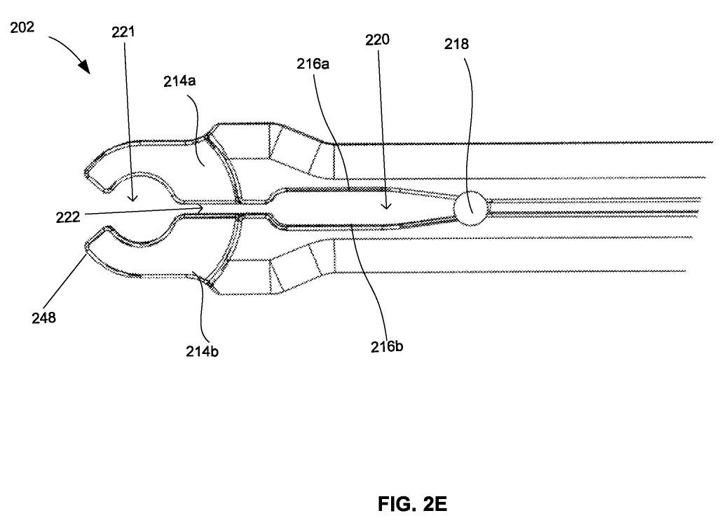

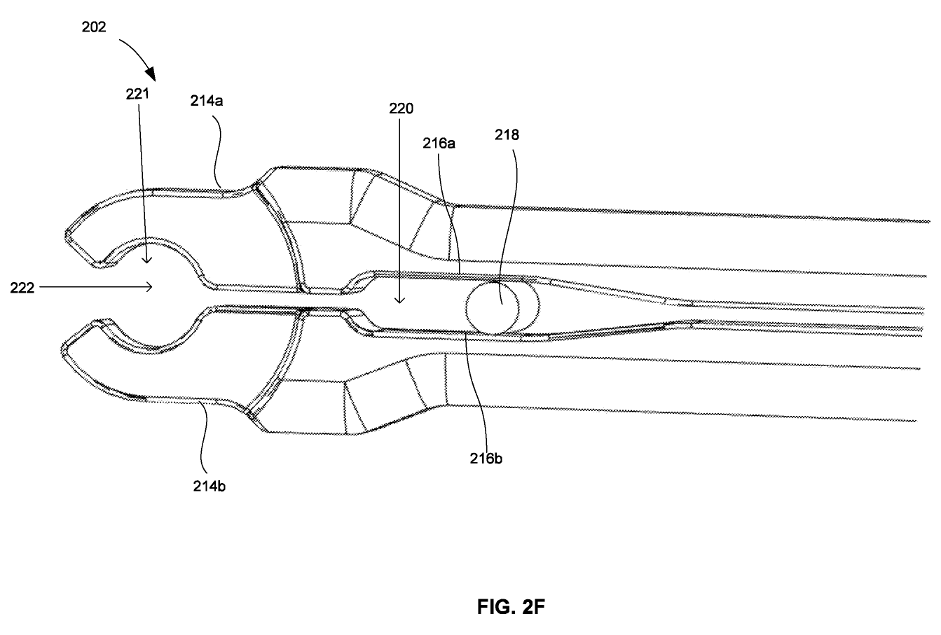

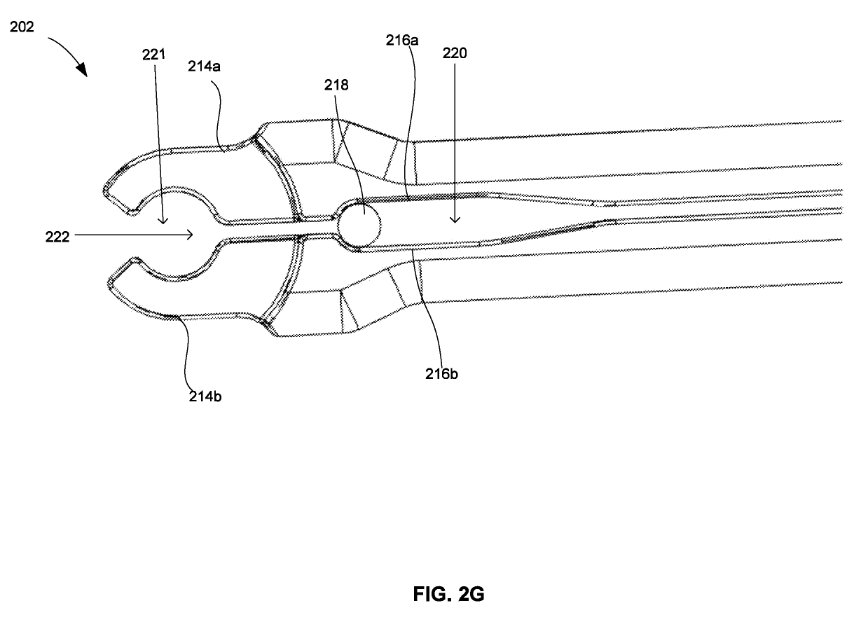

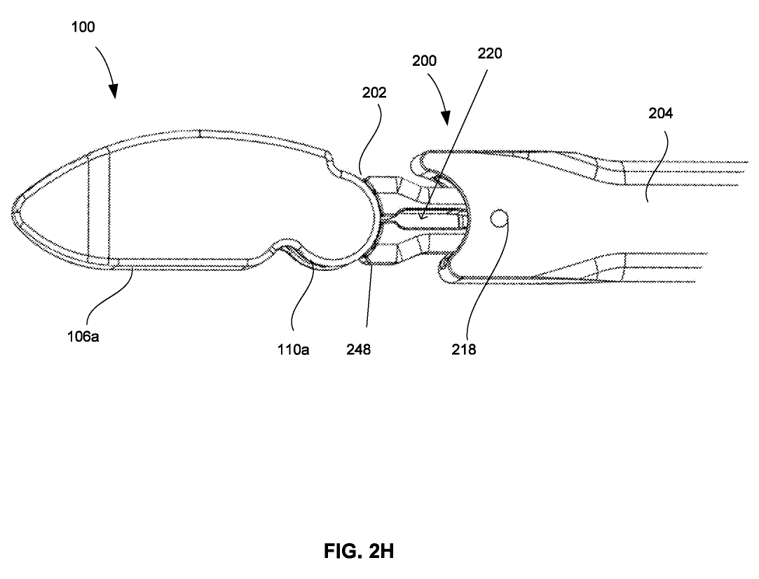

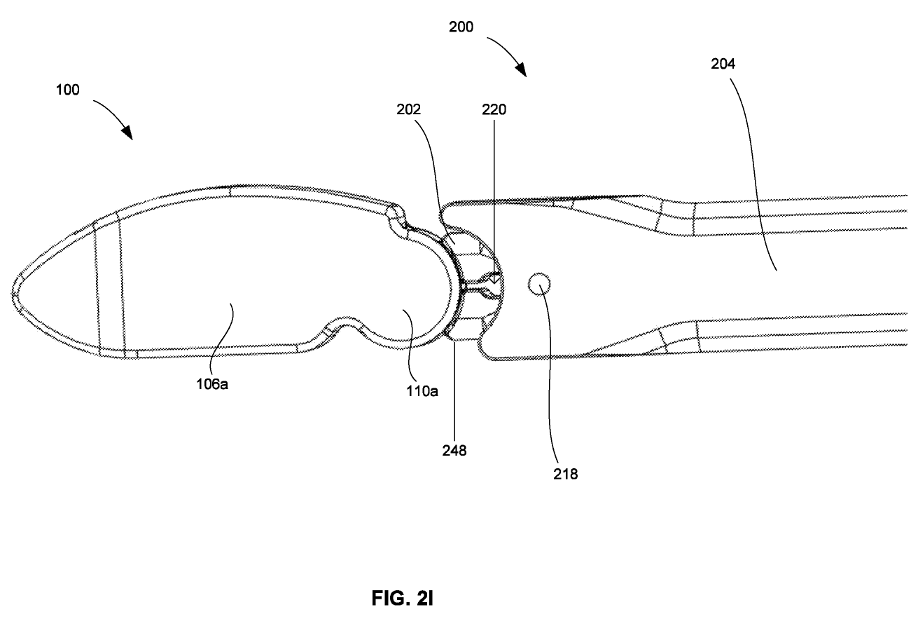

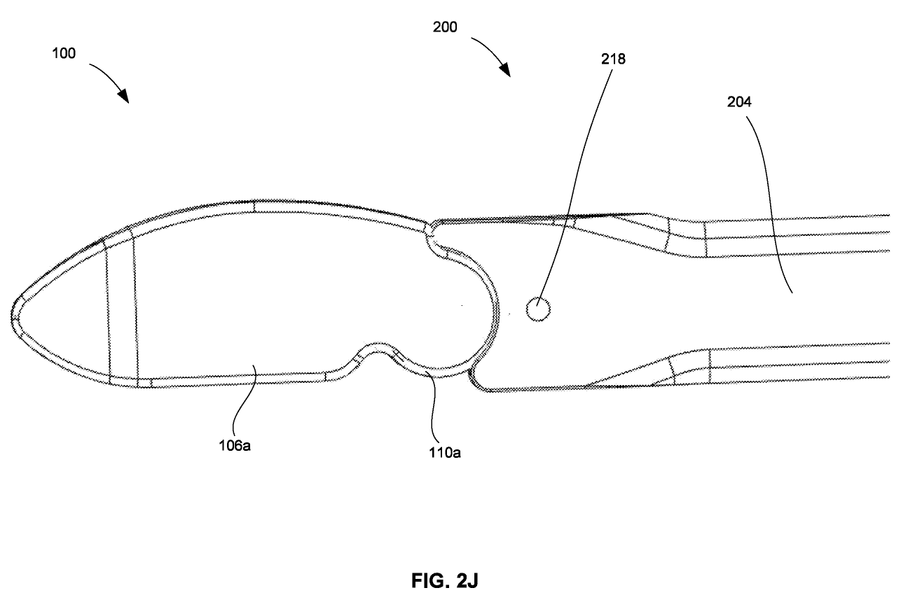

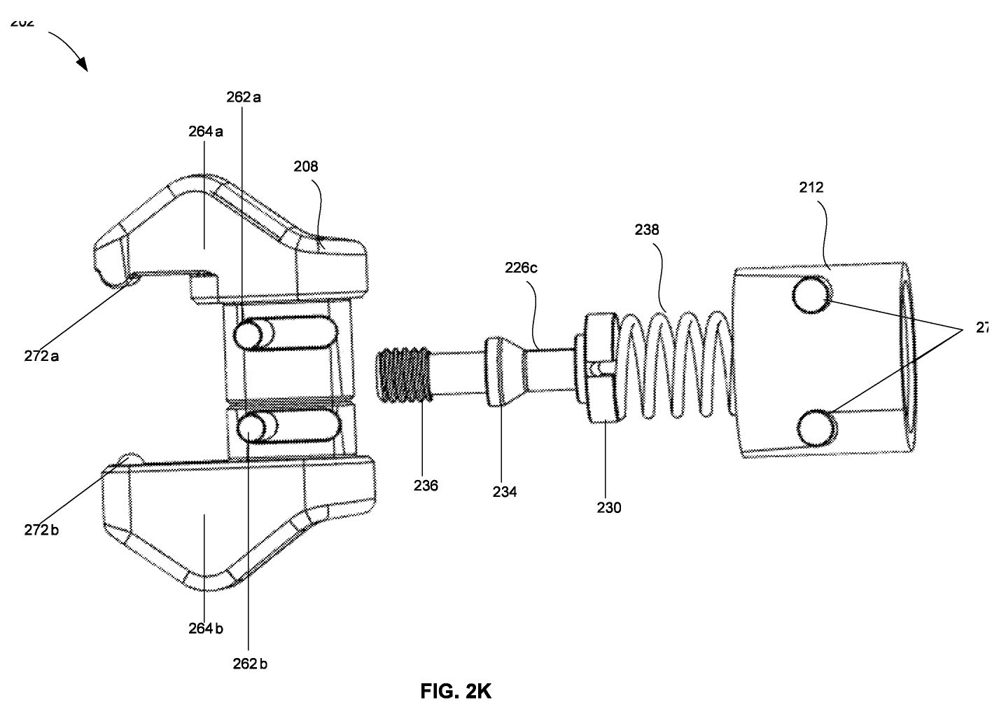

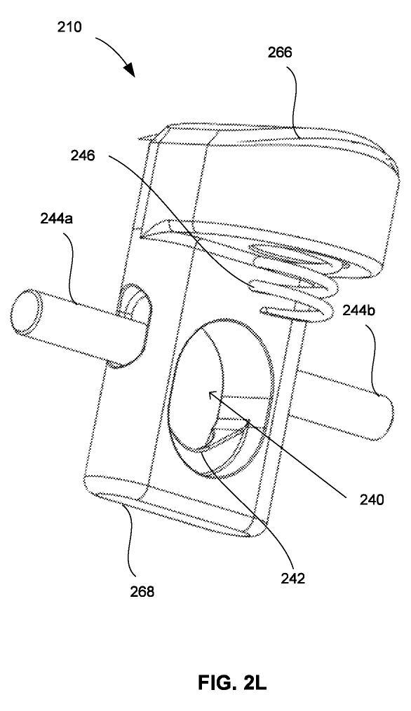

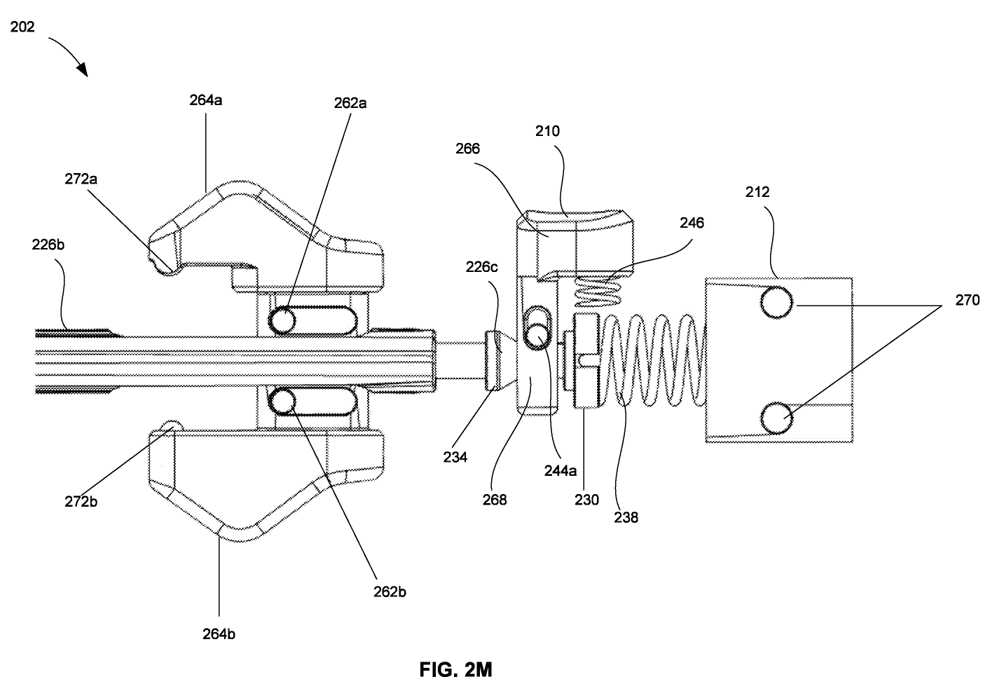

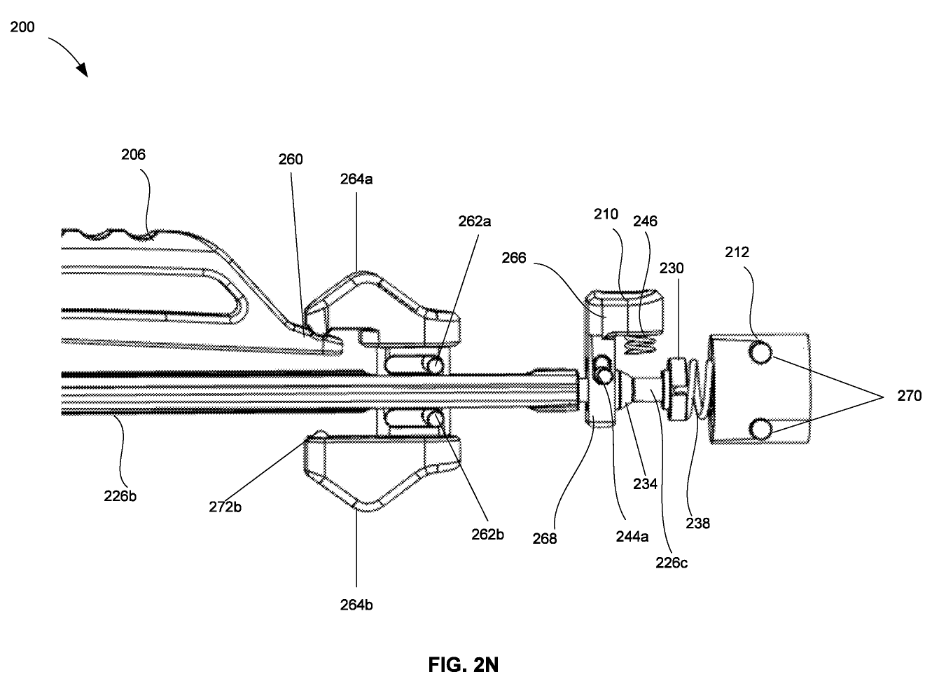

FIGS. 2A-2N depict an example inserter 200, according to an aspect of the present disclosure. Inserter 200 includes a distal end 248 and a proximal end 250. Inserter 200 also includes a first inner shaft 226a including a grasper assembly 202, a second inner shaft 226b connected to first inner shaft 226a, a third inner shaft 226c connected to second inner shaft 226b, an outer shaft 204 surrounding a portion of first inner shaft 226a, and a handle 224 surrounding second and third inner shafts 226b, 226c. Inserter 200 additionally includes a grasping pin 218 connected to outer shaft 204, a lever 206 pivotally connected to first and second inner shafts 226a, 226b, a lever lock 208 on second inner shaft 226b, an unlocking button 210 surrounding third inner shaft 226c, and a slap hammer attachment 212 connected to third inner shaft 226c.

Inserter 200 is capable of three positions: an unlocked position, a semi-locked position, and a locked position. In the unlocked position, an implant can be engaged in grasper assembly 202 or disengaged from grasper assembly 202 without much resistance. In the semi-locked position, an implant engaged in grasper assembly 202 can be rotated about axis X but is otherwise retained by the inserter. In the locked position, an implant engaged in grasper assembly 202 cannot be disengaged or rotated.

As discussed in more detail below, in the unlocked position, when inner shafts 226a, 226b, 226c are extended from outer shaft 204, grasping pin 218 expands grasper assembly 202 to facilitate assembly or disassembly between inserter 200 and an implant, such as implant 100. Additionally, in the locked position, when inner shafts 226a, 226b, 226c are fully retracted, grasper assembly 202 is compressed together by outer shaft 204. Lever 206 contains range of operation to control the amount of compression on an implant, such as implant 100, to either facilitate or prevent rotation of the implant with a user’s single hand. Lever lock 208 locks lever 206 in a locked position to maintain a rotationally locked position.

FIG. 2A depicts lever 206 positioned upward in the unlocked position. A full elevation of lever 206 fully translates inner shafts 226a, 226b, 226c in a distal direction. FIG. 2B depicts lever 206 slightly depressed in the semi-locked position. If starting from an unlocked position, a slight depression of lever 206 slightly translates inner shafts 226a, 226b, 226c in a proximal direction, though inner shafts 226a, 226b, 226c are not translated fully in the proximal direction, like in the locked position. Similarly, if starting from an unlocked position, a slight elevation of lever 206 slightly translates inner shafts 226a, 226b, 226c in a distal direction. FIG. 2C depicts lever 206 fully depressed in the locked position. A full depression of lever 206 fully translates inner shafts 226a, 226b, 226c in a proximal direction.

As shown in FIG. 2D, first, second, and third inner shafts 226a, 226b, 226c are connected. First inner shaft 226a includes a threaded portion (not shown) at its proximal end so that it can be threadedly connected to a distal end of second inner shaft 226b. Similarly, third inner shaft 226c includes a threaded portion 236 at its distal end so that the distal end of third inner shaft 226a can be threadedly connected to a proximal end of second inner shaft 226b. However, in other aspects, the first and third inner shafts can be connected to the second inner shaft through any connection mechanism. Additionally, in other aspects, the inner shaft may be one shaft rather than three connected inner shafts.

Lever 206 enables translation of inner shafts 226a, 226b, 226c in a distal or proximal direction. Also as shown in FIG. 2D, the mechanism of lever 206 is through three connections: the first between handle 224 and lever 206, the second between a fulcrum link 252 and inner shaft 226b, and the third between fulcrum link 252 and lever 206. First, handle 224 connects to the lever 206 by a first pin 254a and a second pin 254d, each received through corresponding holes in handle 224 and second lever 206. Second, fulcrum link 252 connects to inner shaft 226a by a third pin 254b received through corresponding holes in second inner shaft 226b and fulcrum link 252. Third, fulcrum link 252 connects to lever 206 by a fourth pin 254c received through corresponding holes in lever 206 and fulcrum link 252. First pin 254a and second pin 254d are located distal of third pin 254b. A spring 238 abuts a proximal end of third shaft 226c so that inner shafts 226a, 226b, 226c are stabilized during translation. As such, spring 238 is not compressed in the locked position, is partially compressed in the semi-locked position, and is fully compressed in the locked position.

Lever 206 includes a tab 260 at a proximal end, which engages with lever lock 208 to lock lever 206. Lever lock 208 can be translated in a distal direction so as to receive tab 260 of lever 206 between first winged portion 264a of lever lock 208 and handle 224. When lever lock 208 engages tab 260 of lever 206, lever 206 cannot be moved in an upward position. As such, lever lock 208 locks lever 206 in place. Lever lock 208 can only engage tab 260 in the locked position, as detailed below.

Lever 206 also includes an undulated surface 258 and a slit 256 for gripping control. When in use, a user’s fingers may rest on undulated surface 258 with the tips of their fingers received within slit 256.

FIG. 2E depicts grasper assembly 202 of inserter 200 in an unlocked position. Grasper assembly 202 is located at the distal end 248 of first inner shaft 226a. A longitudinal slit 222 defines a first arm 214a and a second arm 214b. Longitudinal slit 222 includes an engagement space 221 between arms 214a, 214b and includes a pin space 220. Engagement space 221 is sized and shaped, for example, to receive post 108 of implant 100 and is defined by rounded ends of first and second arms 214a, 214b. In other aspects, the arms can have any shape of a space that enables grasping of an implant.

Pin space 220 includes grasping pin 218 so that grasping pin 218 can move through pin space 220 when inner shafts 226a, 226b, 226c are translated. Pin space 220 is defined by a concave portion 216a on an internal surface of first arm 214a and a concave portion 216b on an internal surface of second arm 214b. Concave portions 216a, 216b face each other so as to define pin space 220. Pin space 220 is a teardrop shape so that a distance between the arms 214a, 214b at a proximal end of pin space 220 is smaller than a distance between the arms 214a, 214b at a distal end of pin space 220.

Grasping pin 218 is positioned transverse to longitudinal slit 222. Grasping pin 218 is received through two holes on outer shaft 204 so that grasping pin 218 moves through pin space 220 when inner shafts 226a, 226b, 226c are translated.

In the unlocked position, grasping pin 218 is located at a proximal end of pin space 220. Grasping pin 218 can be moved to the proximal end of pin space 220 by translating inner shafts 226a, 226b, 226c in a distal direction so that a distance between the distal end of the grasper assembly 202 and a distal end of outer shaft 204 is greater than in the semi-locked or locked positions. Grasping pin 218 has a diameter slightly larger than a distance between arms 214a, 214b at the proximal end of pin space 220. As such, when grasping pin 218 is at the proximal end of pin space 220, arms 214a, 214b are expanded. Thus, there is sufficient space in engagement space 221 to engage or disengage an implant.

FIG. 2F depicts grasper assembly 202 of inserter 200 in a semi-locked position. Grasping pin 218 is in the middle of pin space 220. Grasping pin 218 can be moved to the middle of pin space 220 by translating inner shafts 226a, 226b, 226c either in a distal or proximal direction, depending on whether inserter 200 is in a locked or unlocked position. Grasping pin 218 has a diameter slightly smaller than a distance between arms 214a, 214b at the middle of pin space 220. However, the distance between arms 214a, 214b at the middle of pin space 220 is larger than the distance at the proximal end. As such, when pin 218 is in the middle of pin space 220, inner shaft 226a does not contact the outer surface of pin space 220, so arms 214a, 214b are in a neutral position. Thus, a distance between the distal end of grasper assembly 202 and a distal end of outer shaft 204 is smaller than in the unlocked position but is not as small as in the locked position. As a result, an implant, such as implant 100, can still be rotated about axis X due to sufficient clearance between the implant and outer shaft 204.

FIG. 2G depicts grasper assembly 202 of inserter 200 in a locked position. Grasping pin 218 is at a distal end of pin space 220. Grasping pin 218 can be moved to the distal end of pin space 220 by translating inner shafts 226a, 226b, 226c in a proximal direction. Grasping pin 218 has a diameter slightly smaller than a distance between arms 214a, 214b at the distal end of pin space 220. As such, when pin 218 is at the distal end of pin space 220, arms 214a, 214b are not expanded and are clamped together due to a positive clamping force by outer shaft 204, as detailed below.

As shown in FIGS. 2H-2J, outer shaft 204 has a curved distal end with the two apexes of the curved lines at points outside of grasper assembly 202. The curved lines of outer shaft 204 complement the shape of an implant, such as the portion of implant 100 shaped by first concave portion 118a, convex portion 120, and second concave portion 118b on implant 100. This permits fluid rotation of implant 100 with respect to inserter 200.

As shown in FIG. 2H depicting inserter 200 in an unlocked position, and as shown in FIG. 2I depicting inserter 200 in a semi-locked position, the position of outer shaft 204 may not limit rotation of an implant, as is the case with implant 100. However, as shown in FIG. 2J, the position of outer shaft 204 may prevent rotation of an implant, thus securing the implant. For example, when the implant is implant 100, implant 100 cannot be rotated because the shape formed by first concave portion 118a, convex portion 120 abuts the curved ends of outer shaft 204. In this position, a distance between the distal end of grasper assembly 202 and a distal end of outer shaft 204 is smaller compared to the unlocked and semi-locked positions.

As shown in FIG. 2K, the proximal end of inserter 200 includes a slap hammer attachment 212. Slap hammer attachment 212 is indirectly connected to a flat portion 230 of third inner shaft 226c through spring 238. Slap hammer attachment 212 is connected to handle 224 through two pins 270 in corresponding holes of slap hammer attachment 212 and handle 224. Slap hammer attachment 212 may be internally threaded to receive, for example, a slap hammer.

As shown in FIG. 2K-2N, third inner shaft 226c includes the flat portion 230 at a proximal end and a fin 234 at a distal end. Fin 234 has a ramped surface that tapers to its proximal end so that unlocking button 210 can engage with fin 234 when compressed.

Such compression of unlocking button 210 only occurs when inserter 200 is in the locked position. Though unlocking button 210 can be depressed in any position (unlocked, semi-locked, or locked), fin 234 only fully passes through hole 240 in the locked position and thus only serves a functional purpose in the locked position.

Unlocking button 210 is positioned on third inner shaft 226c. Unlocking button 210 is L-shaped including a top portion 266 being a compressible button and a side portion 268 through which third inner shaft 226c is received. Specifically, top portion 266 includes a spring 246, which, as stated above, can be compressed when inserter 200 is a locked position. Side portion 268 includes a pin 244 received within handle 224 to connect unlocking button 210 to handle 224. So that handle 224 is not moved when the unlocking button 210 is compressed, side portion 268 includes a slit in which pin 244 is received. As such, pin 244 do not move when unlocking button 210 is compressed.

Additionally, side portion 268 includes a hole 240 through which third inner shaft 226c is received. The bottom of hole 240 includes a barrier 242, which abuts fin 234 of third inner shaft 226c when the inserter 200 is in a locked position. When inserter 200 is in an unlocked position, unlocking button 210 is positioned at a proximal end of third inner shaft 226c so that spring 246 of unlocking button 210 is positioned over flat portion 230. As such, unlocking button 210 cannot be compressed in the unlocked position.

Unlocking button 210 is positioned distally of fin 234. As such, in a non-compressed position of unlocking button 210, barrier 242 abuts fin 234. In a compressed position of unlocking button 210, unlocking button 210 is pressed downward, which also moves barrier 242 downward so that barrier 242 does not abut fin 234. The downward position of barrier 242 enables clearance for fin 234 to be received through hole 240 of unlocking button 210. As such, inner shafts 226a, 226b, 226c translate in a distal direction. This movement of inner shafts 226a, 226b, 226c shifts inserter 200 from being in a locked position to being in an unlocked position. However, if lever lock 208 is engaged, unlocking button 210 will not cause such translation of inner shafts 226a, 226b, 226c. When unlocking button 210 is compressed, spring 246 provides some resistance to keep unlocking button 210 in place.

As shown in FIG. 2N depicting inserter 200 in a locked position, lever lock 208 includes two protrusions 272a, 272b that are positioned on either side of second inner shaft 226b so that lever lock 208 can translate along second inner shaft 226b. Lever lock 208 additionally includes two winged portions 262a, 262b on either side of second inner shaft 226b to engage with tab 260 of lever 206. Each winged portion 262a, 262b includes an internally facing protrusion 272a, 272b. Protrusion 272a engages with lever 206, and protrusion 272b engages with handle 224 to hold lever lock 208 in place. Such engagement only occurs when inserter 200 is in the locked position.

A method of using inserter 200 includes positioning lever 206 to an upward position to allow for assembly with an implant, such as implant 100. In this unlocked position, inner shafts 226a, 226b, 226c are translated distally such that pin 218 is at the proximal end of pin space 220 so that arms 214a, 214b are expanded from their neutral position, allowing implant 100 to be engaged in the engagement space 221 between the rounded ends of arms 214a, 214b.

Lever 206 is then partially depressed so that inner shafts 226a, 226b, 226c partially translate proximally into a semi-locked position such that pin 218 is at the middle of pin space 220 and arms 214a, 214b are in a neutral position. In this position, implant 100 can still be rotated about axis X due to sufficient clearance between the implant 100 and outer shaft 204.

Once implant 100 is adjusted to a desired orientation, lever 206 is fully depressed into a locked position so that inner shafts 226a, 226b, 226c fully translate proximally for a locked position such that pin 218 is at the distal end of pin space 220 and arms 214a and such that outer shaft 204 compressed arms 214a, 214b. As such, implant 100 is be secured within compressed arms 214a, 214b. Additionally, the shape formed by first concave portion 118a, convex portion 120, and second concave portion 118b abuts the curved ends of outer shaft 204 so that implant 100 cannot be rotated. Thus, implant 100 is stabilized in the desired position.

When inserter 200 is in the locked position, implant 100 is inserted between two vertebrae so that implant 100 can be articulated into a desired orientation. A slap hammer can be optionally coupled to slap hammer attachment 212 so that a slap hammer can be used to insert the implant 100 into a patient.

Lever 206 is moved into a semi-locked or locked position from the locked position as needed, for example, to further adjust an orientation of implant 100. As such, implant 100 can thus be manipulated by securing implant 100 in a semi-locked position and rotating implant 100 as needed by switching between the locked and semi-locked positions.

At any time when inserter 200 is in a locked position, to further lock inserter 200, lever lock 208 can be translated in a distal direction so as to receive tab 260 of lever 206 between first winged portion 264a of lever lock 208 and handle 224. As such, lever 206 is not be moveable in an upward position.

At any time when inserter 200 is in a locked position, to unlock inserter 200, unlocking button 210 is pressed downward, which also moves barrier 242 downward so that barrier 242 does not abut fin 234. The downward position of barrier 242 enables clearance for fin 234 to be received through hole 240 of unlocking button 210. As such, inner shafts 226a, 226b, 226c translate in a distal direction. However, if lever lock 208 is engaged, unlocked button 210 will not cause such translation of inner shafts 226a, 226b, 226c.

To disengage implant 100 from the inserter, lever 206 is moved fully in an upward direction for an unlocked position.

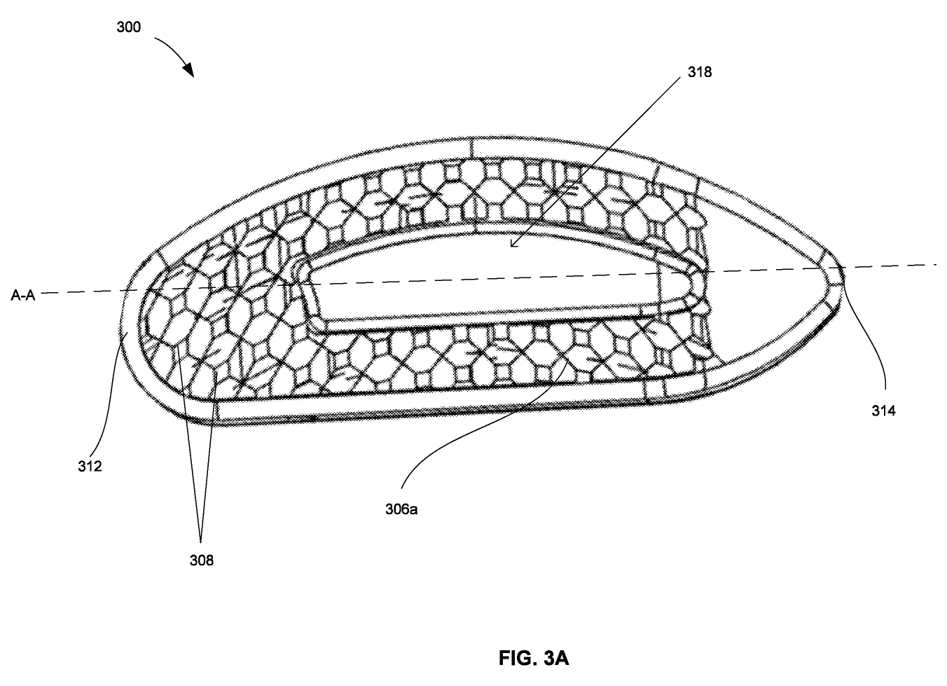

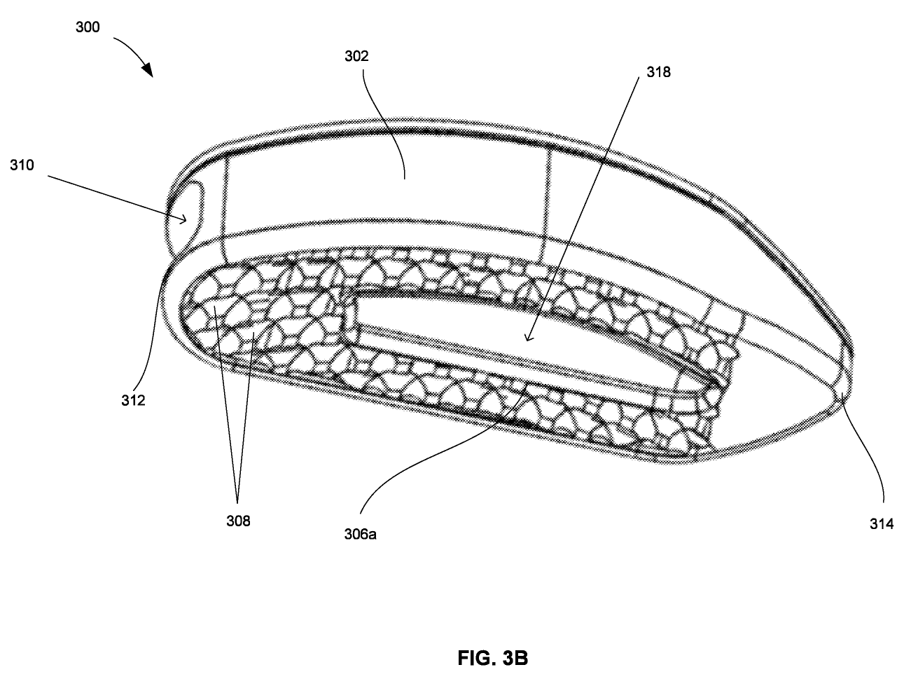

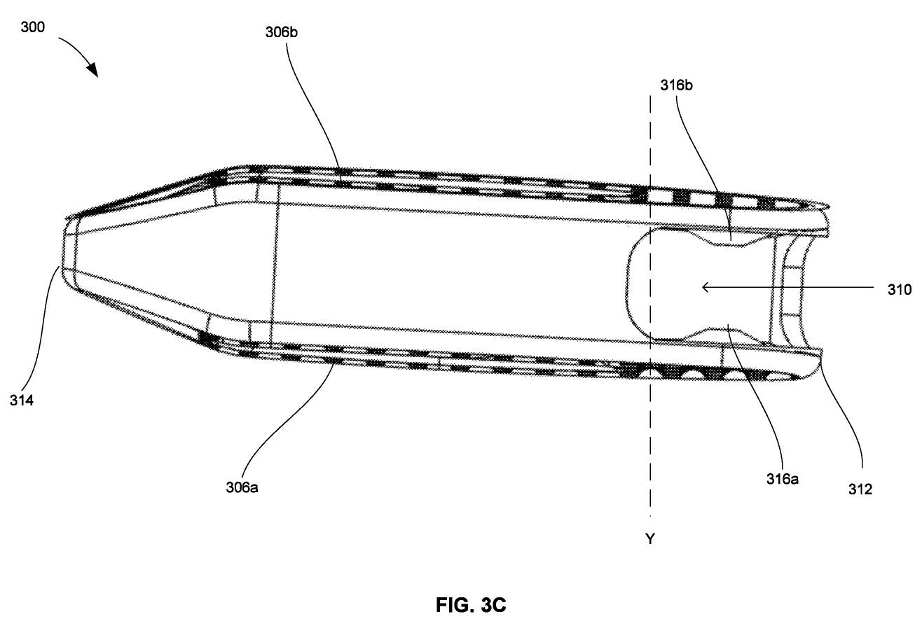

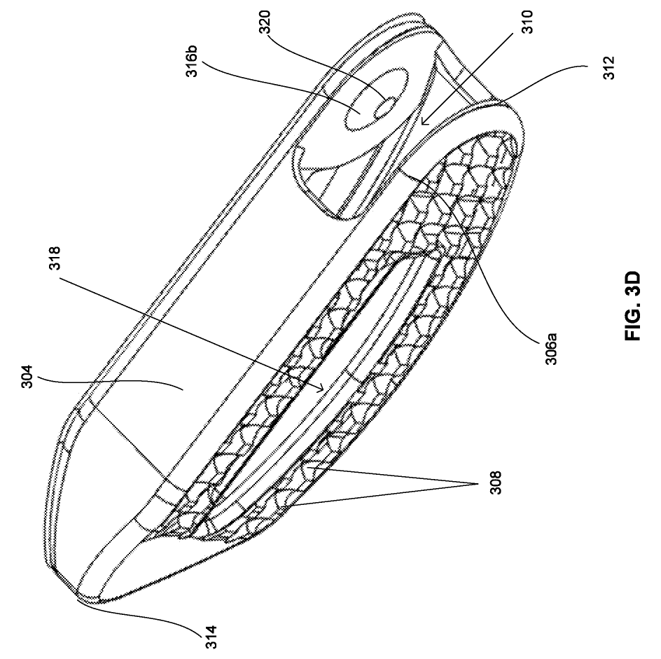

FIGS. 3A-3D depict an implant 300, according to another aspect of the present disclosure. Implant 300 includes a first lateral side 302, a second lateral side 304, a superior side 306a, an inferior side 306b, a posterior end 312, and an anterior end 314. Implant 300 includes protrusions 308 on superior and inferior sides 306a, 306b, a window 318 through superior and inferior sides 306a, 306b, and a recess 310 on posterior end 312.

The outer configuration of implant 300 has a generally curved, kidney bean-like shape, and thus is consistent with the shapes of interbody implants used in TLIF techniques. However, the shape of implant 300 can vary depending upon the particular use for the implant. Protrusions 308 on superior and inferior sides 306a, 306b are pyramidal with a flat top surface. However, the protrusions on implant 300 can be any size and shape that can engage a bone. Window 318 through superior and inferior sides 306a, 306b is a substantially oval shape to receive, for example, bone graft material. However, in other aspects, implant 300 may include any number of windows of any size and shape on any side of the implant body. Additionally, in other aspects, implant 300 may not include any windows.

As shown in FIGS. 3C-3D, recess 310 on posterior end 312 includes a first convex portion 316a on superior side 306a and a second convex portion 316b on inferior side 306b. Each convex portion 316a, 316b includes a flat portion 320. Flat portion 320 a flat horizontal surface that is parallel to the center plane of 304. First and second convex portions 316a, 316b are symmetrical so as to define a keyhole shape. First and second convex portions 316a, 316b are intended to be engaged by inserter 400, as described below.

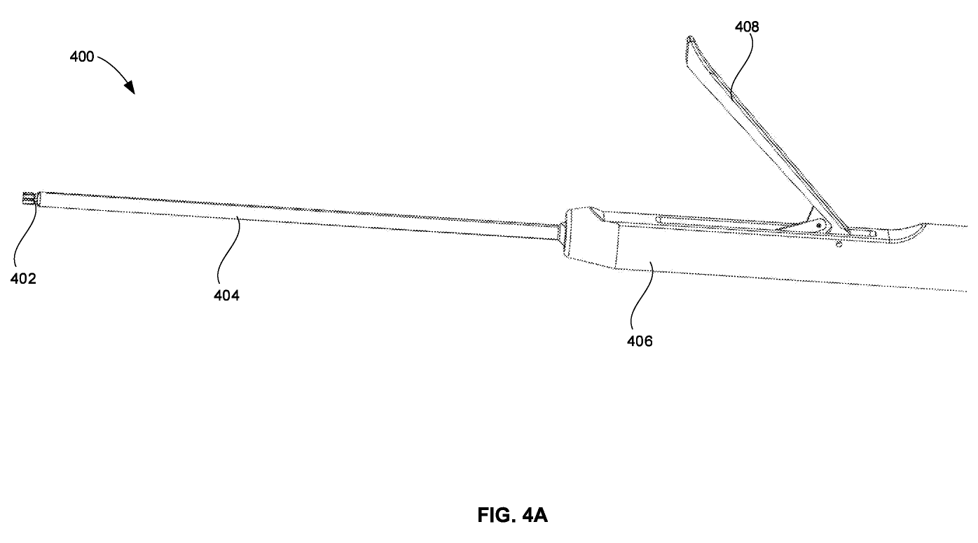

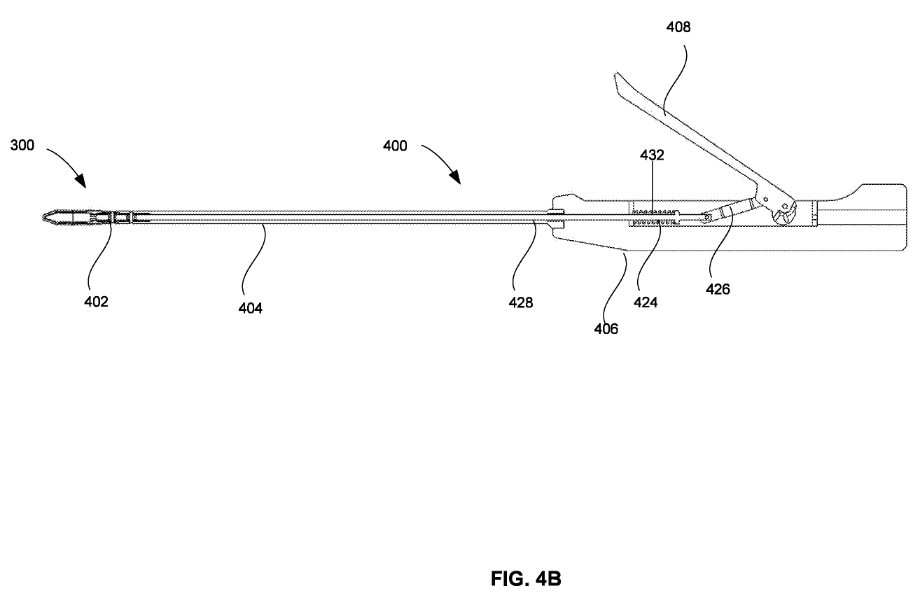

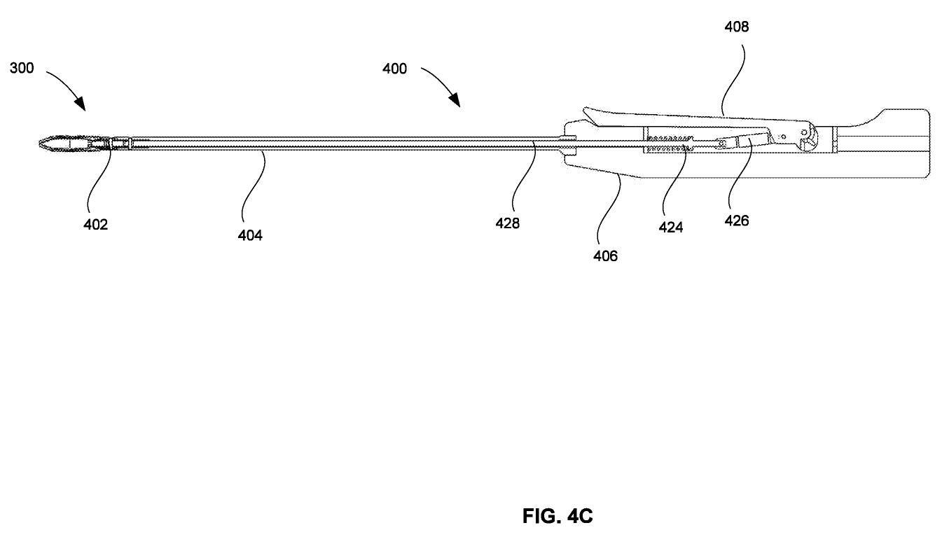

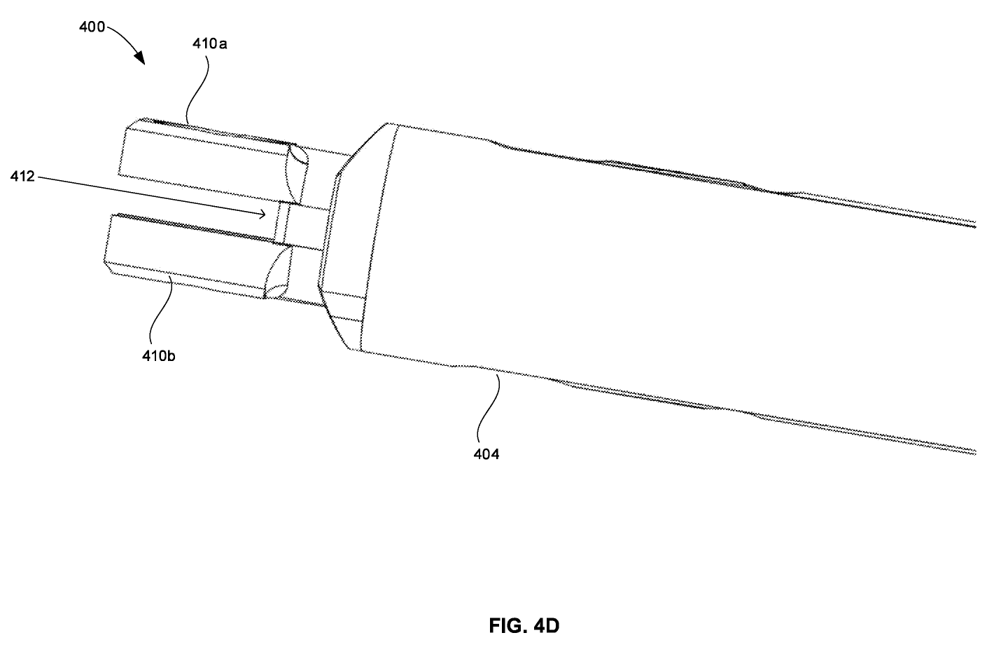

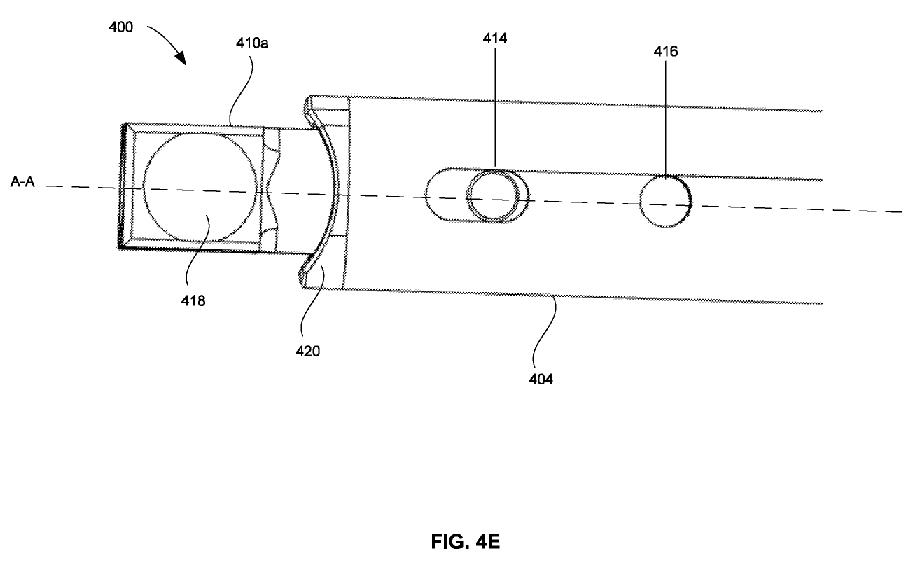

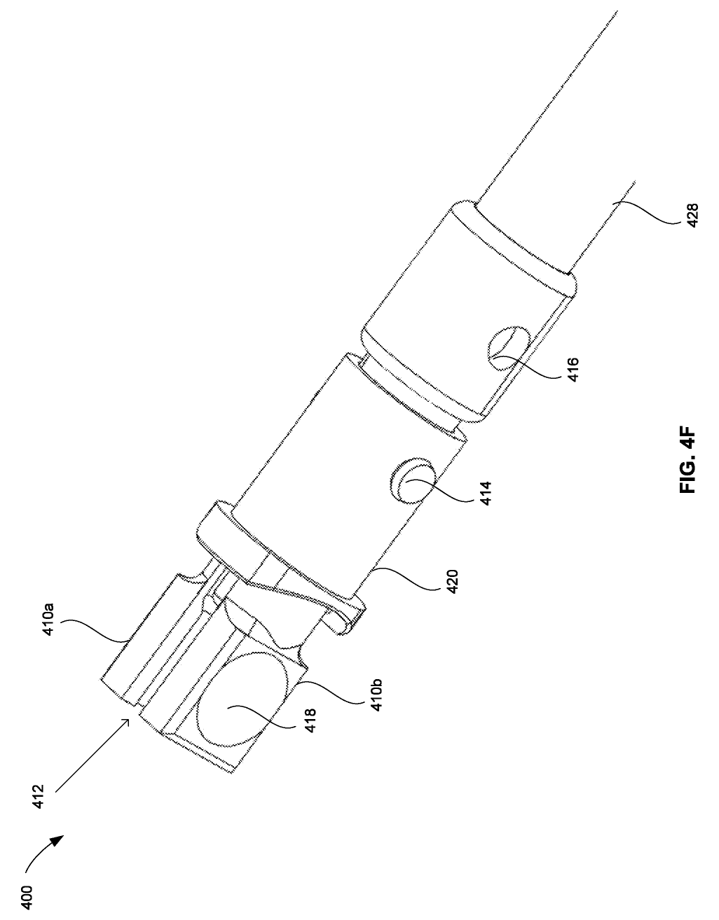

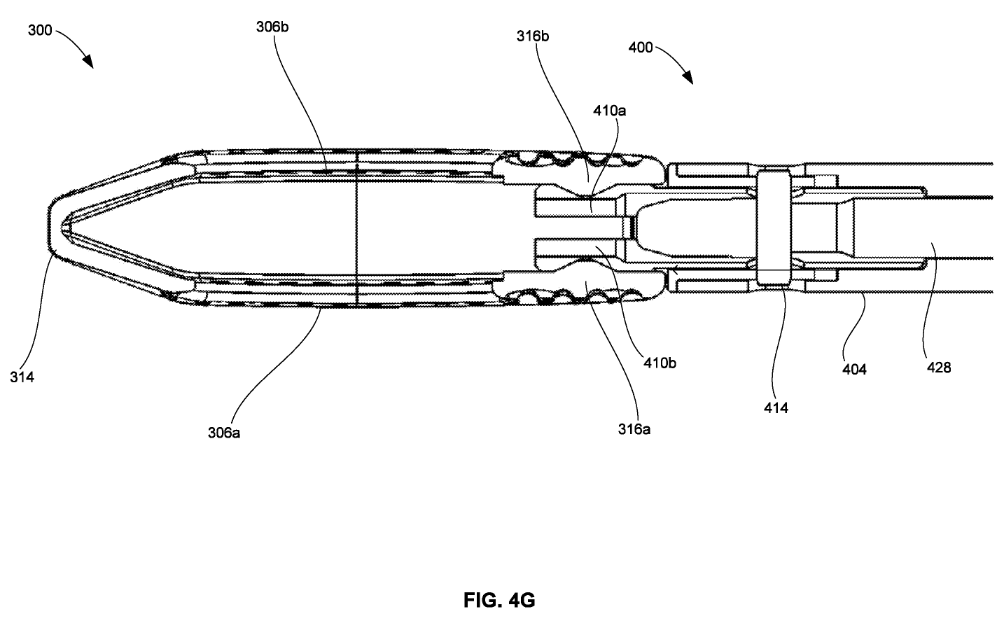

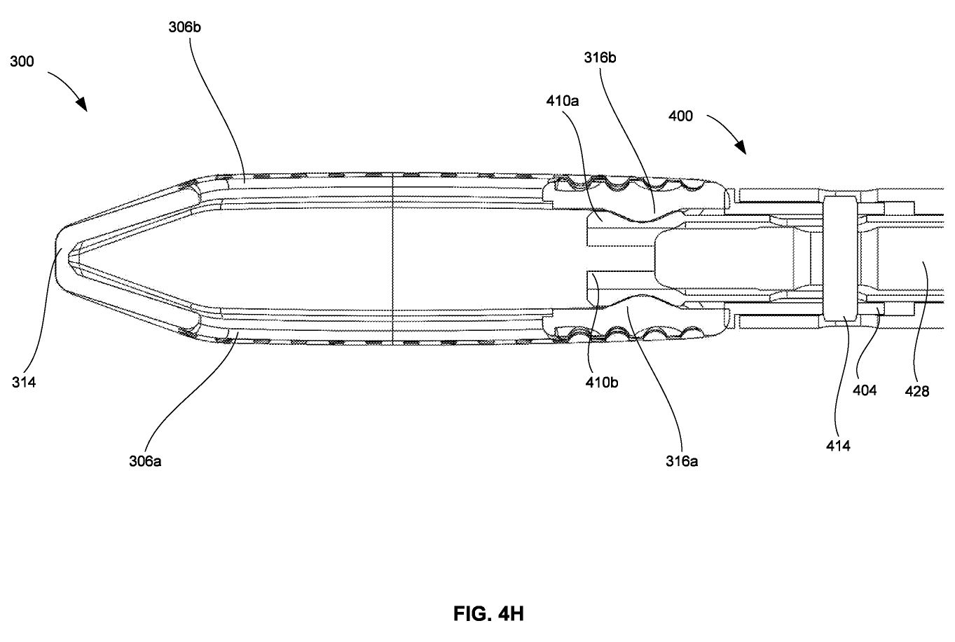

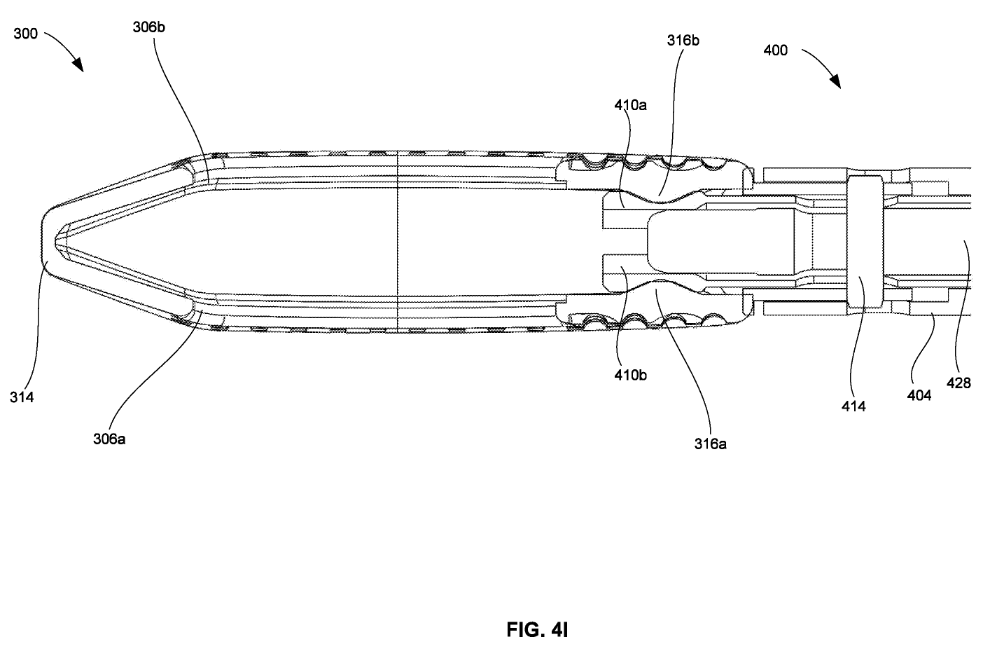

FIGS. 4A-4I depict an example inserter 400, according to an aspect of the present disclosure. Inserter 400 includes an anterior end 448 and a posterior end 450 of inserter 400 are labeled. Inserter 400 also includes a shaft 428 translatable by a lever 408, a grasper assembly 402 on a distal end of shaft 428, an outer shaft 404 surrounding shaft 428 and a portion of grasper assembly 402, and a handle 406 surrounding lever 408.

Inserter 400 is capable of three positions: an unlocked position, a semi-locked, and a locked position. In the unlocked position, an implant engaged in grasper assembly 402 or disengaged from grasper assembly 402 without much resistance. In the semi-locked position, an implant engaged in grasper assembly 402 can be rotated about axis Y. In the locked position, an implant engaged in grasper assembly 402 cannot be disengaged or rotated.

FIG. 4A depicts lever 408 positioned upward in the locked position. A full elevation of lever 408 fully translates shaft 428 in a distal direction. FIG. 4B depicts lever 408 slightly depressed in the semi-locked position. If starting from an unlocked position, a slight depression of lever 408 slightly translates shaft 428 in a proximal direction, though shaft 428 are not translated fully in the proximal direction, like in the locked position. Similarly, if starting from an unlocked position, a slight elevation of lever 408 slightly translates shaft 428in a distal direction. FIG. 4C depicts lever 408 in a fully depressed in the locked position. A full depression of lever 408 fully translates shaft 428in a proximal direction

As shown in FIGS. 4B-4C, shaft 428 is pivotally connected to a fulcrum link 426, which is pivotally attached to lever 408. Shaft 428 includes a protrusion 432 at a proximal portion so that a spring 424 is positioned between a distal surface of protrusion 432 and a surface of handle 406. As lever 408 is moved in a downward position, shaft 428 is translated in a distal direction, and spring 424 is compressed. In the unlocked position, lever 408 is in an upward position, and spring 424 is not compressed.

As shown in FIG. 4D, a longitudinal slit 412 in grasper assembly 402 defines a first arm 410a and a second arm 410b. Arms 410a, 410b are straight. As shown in FIG. 4E, arms 410a and second arm 410b are anchored to outer shaft 404 by a pin 416.

As shown in FIGS. 4E-4F, first and second arms 410a, 410b each include a concave portion 418 on its outer surface. Concave portion 418 also includes a flat horizontal surface (not shown) parallel to longitudinal slit 412. Each concave portion 418 is sized and shaped to receive convex portions of an implant, such as convex portions 316a, 316b of implant 300. When convex portions 316a, 316b of implant 300 are mated with concave portion 418 of inserter 400, the flat horizontal surface (not shown) of inserter 400 mates with flat portion 320 of implant 300.

As shown in FIGS. 4G-4I, grasper assembly 402 is received over a distal end of shaft 428. A collar 420 is positioned over a portion of grasper assembly 402. A pin 414 is received in shaft 428, within a longitudinal slot (not shown) in grasper assembly 402, and in collar 420 to connect collar 420 to shaft 428. As such, when shaft 428 is translated, collar 420 is translated in the same direction, but grasper assembly 402 does not translate.

As shown in FIG. 4G, in the unlocked position, concave portions 418 on first and second arms 410a, 410b are received in convex portions 316a, 316b of implant 300. Shaft 428 is not received between convex portions 316a, 316b so that first and second arms 410a, 410b are not compressed against convex portions 316a, 316b.

As shown in FIG. 4H, in the semi-locked position, shaft 428 is partially received between convex portions 316a, 316b so that first and second arms 410a, 410b are partially compressed against convex portions 316a, 316b. Shaft 428 does not fully compress the arms 410a, 410b in this position because shaft 428 is not positioned past an apex of each convex portion 316a, 316b. To translate shaft 428 in a distal direction for the semi-locked position, lever 408 is partially depressed in a downward position from an unlocked position or partially moved in an upward position from a locked position.

As shown in FIG. 4I, in the locked position, shaft 428 is fully received between convex portions 316a, 316b so that first and second arms 410a, 410b are fully compressed against convex portions 316a, 316b. Shaft 428 fully compresses arms 410a, 410b in this position because shaft 428 applies force on arms 410a, 410b to the apex of each convex portion 316a, 316b. To translate shaft 428 in a distal direction for the locked position, lever 408 is depressed in a downward position from a semi-locked position. Additionally, when implant 300 is engaged, a distal edge of collar 420 abuts a proximal end of implant 300 and thus locking implant 300 in place.

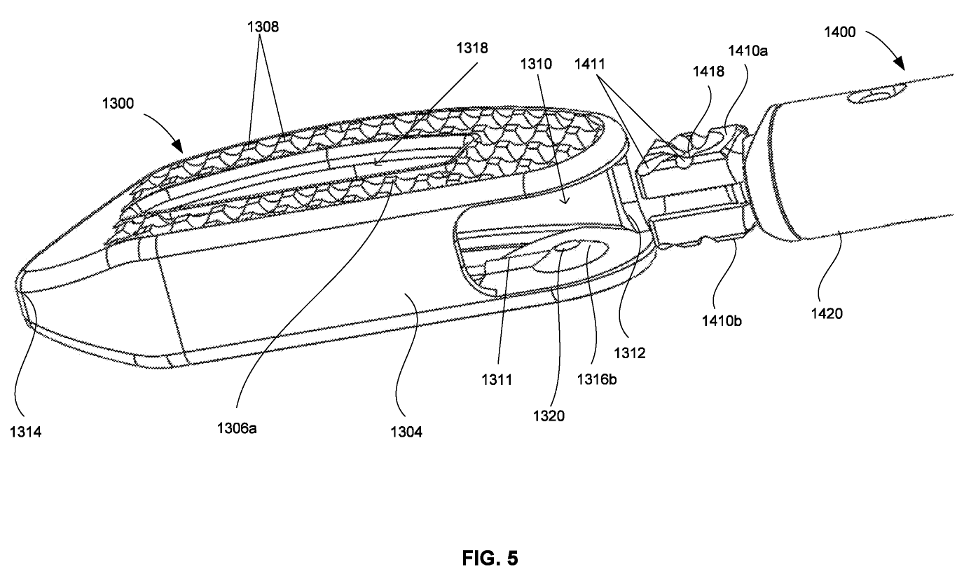

FIG. 5 depicts an example inserter 1400 and an example implant 1300, according to an aspect of the present disclosure. In this aspect, inserter 1400 is similar to inserter 400 in FIGS. 4A-4I, and therefore like elements are referred to with similar numerals within the 1400-series of numbers. Similarly, implant 1300 is similar to implant 300 in FIGS. 3A-3D, and therefore like elements are referred to with similar numerals within the 1300-series of numbers. The description of certain similar features between inserter 1400 and inserter 400 and between implant 1300 and implant 300 is omitted for brevity, with the following focusing on the differences between the two inserters and implants.

Implant 1300 differs in that it further includes a raised surface 1311 extending from an anterior end of convex portion 1316b to recess 1310. Raised surface 1311 has a uniform width, but in other aspects, the raised surface may increase or decrease in width along its length. Similarly, although shown extending from an anterior end of convex portion 1316b, raised surface 1311 can extend from any portion thereof or may even be situated entirely separate from the convex portion.

Inserter 1400 differs in that it further includes grooves 1411 radially extending from concave portion 1418 to an outer edge of each arm 1410a,1410b. Though FIG. 5 depicts three grooves 1411, in other aspects, there may be any number of grooves. Grooves 1411 have a uniform width, but in other aspects, the grooves may increase or decrease in width along its length. Like with raised surface 1311, grooves 1411 can be positioned differently and may not extend entirely to an outer edge of each arm 1410a, 1410b. The inclusion of different numbers of grooves permits different angulations of the implant with respect to the inserter to be achieved. Because of the below-discussed cooperation of raised surface 1311 and grooves 1411, such structures can be modified from what is shown, but in a manner in which they still are capable of cooperating with one another.

Grooves 1411 of inserter 1400 are sized and shaped to receive raised surface 1311 of implant 1300. Each groove 1411 corresponds to a different angulation of inserter 1400 relative to implant 1300. When one of the grooves 1411 is engaged with raised surface 1311, implant 1300 is prevented from rotational movement relative to inserter 1400. Such engagement improves the connection between the inserter 1400 and implant 1300 and thereby improves user manipulation of implant 1300 during insertion into a patient. Of course, disengagement of raised surface 1311 and grooves 1411 will permit rotational movement of implant 1300 with respect to inserter 1400, as discussed elsewhere herein.

In other aspects, the implant may instead include a groove, and the inserter may instead include raised portions that are each configured to be received within the groove of the implant.

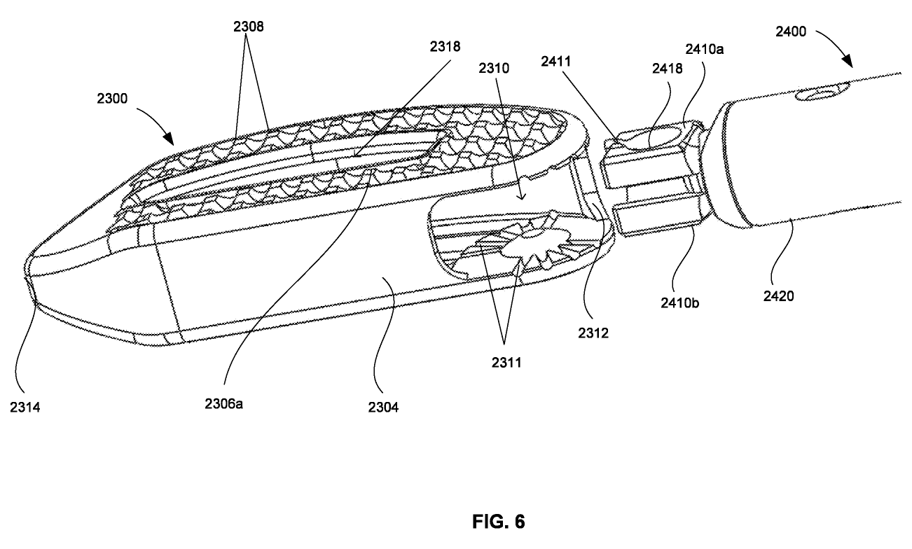

For instance, FIG. 6 depicts an example inserter 2400 and an example implant 2300, according to another aspect of the present disclosure. In this aspect, inserter 2400 is similar to inserter 1400 in FIG. 5, and therefore like elements are referred to with similar numerals within the 2400-series of numbers. Similarly, implant 2300 is similar to implant 1300 in FIG. 5, and therefore like elements are referred to with similar numerals within the 2300-series of numbers. The description of certain similar features between inserter 2400 and inserter 1400 and between implant 2300 and implant 1300 is omitted for brevity, with the following focusing on the differences between the two inserters and implants.

Implant 2300 differs in that it does not include a raised surface, such as raised surface 1311. Instead, implant 2300 includes grooves 2311 radially extending outward from a circumference of convex portion 316b. Though FIG. 6 depicts eight grooves 2311, in other aspects, there may be any number of grooves. Grooves 2311 each have a uniform width, but in other aspects, the each groove may increase or decrease in width along its length.

Inserter 2400 differs in that it does not include grooves, such as grooves 1411. Instead, inserter 2400 includes a raised surface 2411 extending from a circumference of convex portion 316b to an anterior end of inserter 2400. Raised surface 2411 has a uniform width, but in other aspects, the raised surface may increase or decrease in width along its length.

Grooves 2311 of implant 2300 are sized and shaped to receive raised surface 2411 of inserter 2400. Each groove 2311 corresponds to a different angulation of inserter 2400 relative to implant 2300. When one of the grooves 2311 is engaged with raised surface 2411, implant 2300 is prevented from rotational movement relative to inserter 2400. Such engagement improves the connection between the inserter 2400 and implant 2300 and thereby improves user manipulation of implant 2300 during insertion into a patient. Again, disengagement will permit rotation to occur.

In other aspects, the implant may instead include raised portions, and the inserter may instead include a groove configured to receive the raised portion of the implant.

A method of using inserter 400-2400 includes moving lever 408-2408 to an upward position to allow for assembly of an implant, such as implant 300-2300. With inserter 400-2400 in an unlocked position, grasper assembly 402-2402 is inserted into recess 310-2310 of implant 300-2300 so that concave portions 418-2418 on first and second arms 410a,b-2410a,b of grasper assembly 402-2402 are received in convex portions 316a,b-2316a,b of implant 300-2300. With lever 408-2408 in an upward position, shaft 428-2428 is not received between convex portions 316a,b-2316a,b so that first and second arms 410a,b-2410a,b are not compressed against convex portions 316ab-2316a,b. For inserter 1400, one of the grooves 1411 of inserter 1400 receives raised surface 1311 of implant 1300 based on a desired angulation of inserter 1400 relative to implant 1300. For inserter 2400, one of the grooves 2311 of implant 2300 receives raised surface 2411 of inserter 2400 based on a desired angulation of inserter 2400 relative to implant 2300.

Implant 300-2300 is inserted between two vertebrae so that implant 300-2300 can be articulated into a desired orientation. Lever 408-2408 is then partially depressed so that shaft 428-2428 is partially received between convex portions 316ab-2316a,b and so that first and second arms 410a,b-2410a,b are in a neutral position. With inserter 400-2400 in the semi-locked position, implant 300-2300 is rotated about axis Y until implant 300-2300 is in a desired orientation. Once implant 300-2300 is adjusted to a desired orientation, lever 408-2408 is fully depressed for a locked position so that shaft 428-2428 is fully received between convex portions 316a,b-2316a,b and so that first and second arms 410a,b-2410a,b are fully compressed against convex portions 316ab, 2316a,b. With inserter 400-2400 in the locked position, implant 300-2300 is stabilized in the desired position.

Lever 408-2408 is moved upward into a semi-locked or locked position from the locked position as needed, for example, to further adjust an orientation of implant 300-2300. As such, implant 300-2300 can thus be manipulated by securing implant 300-2300 in a semi-locked position and rotating implant 300-2300 as needed by switching between the locked and semi-locked positions.

To disengage implant 300-2300, lever 408-2408 is moved fully in an upward direction for an unlocked position.

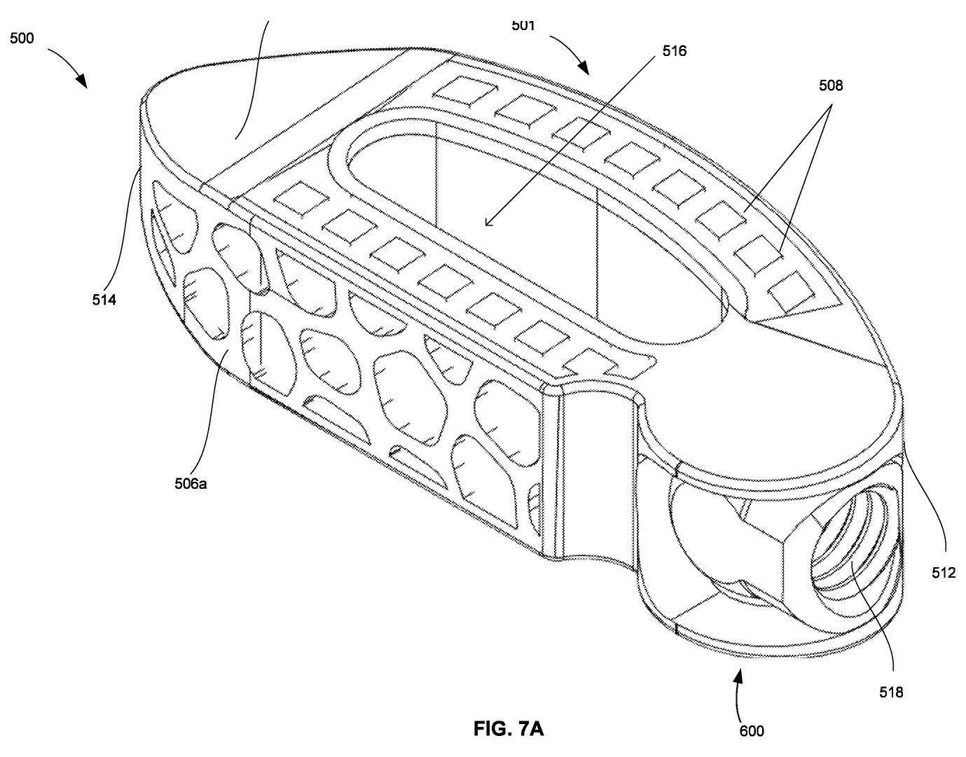

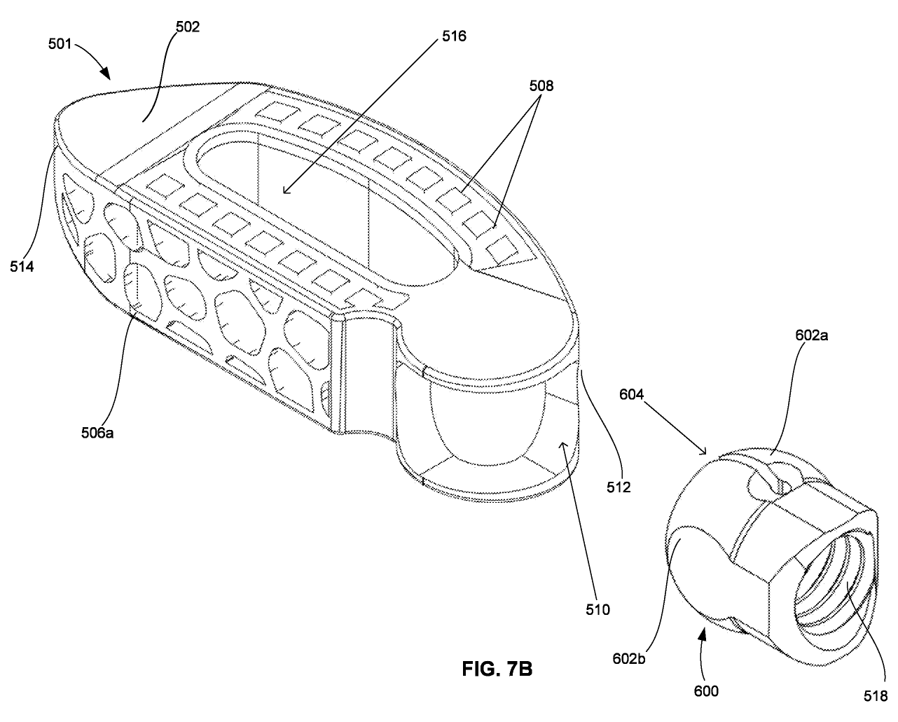

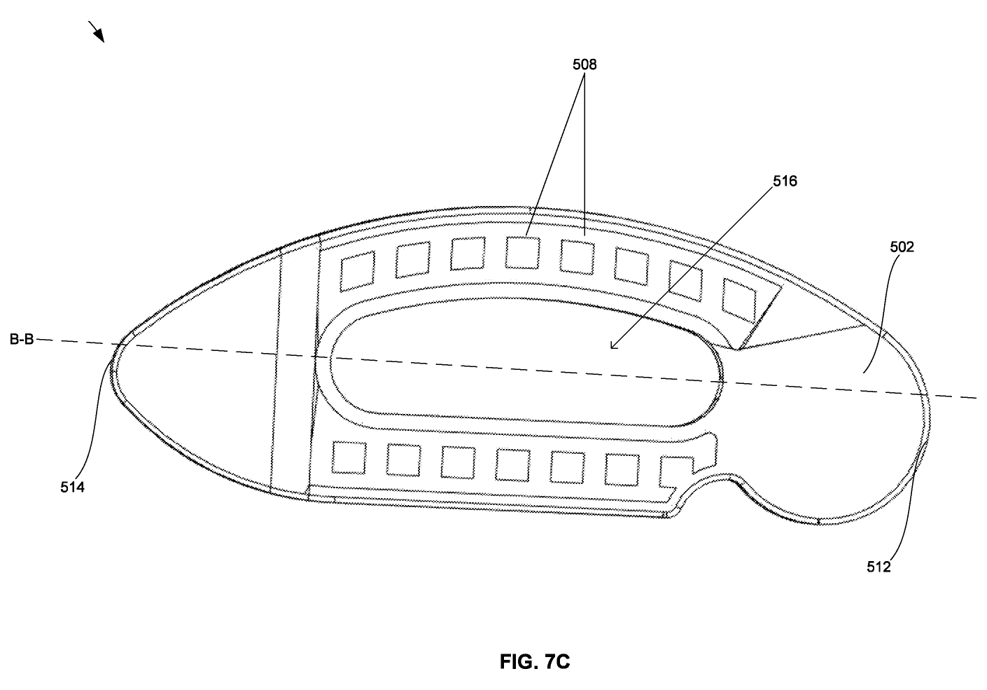

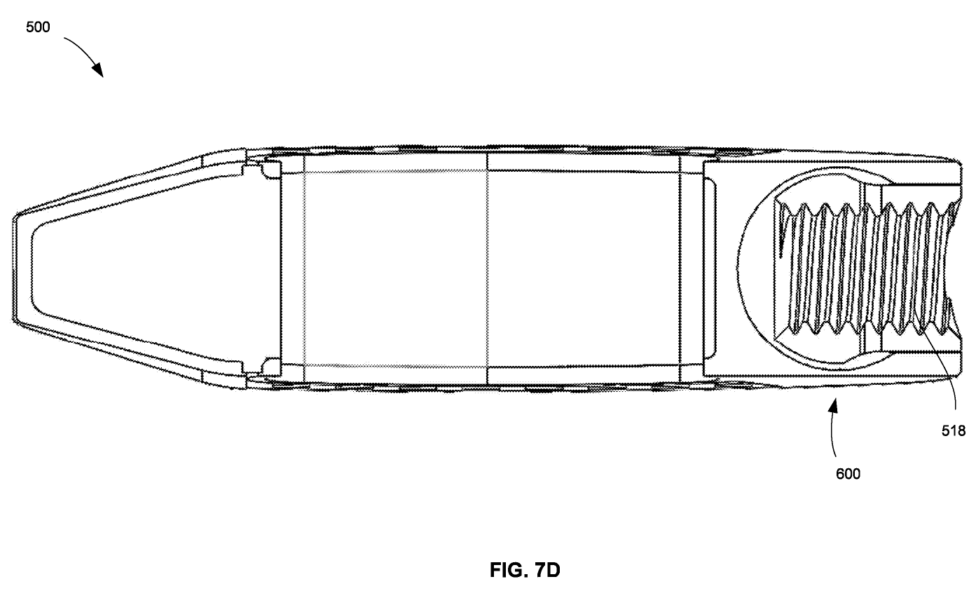

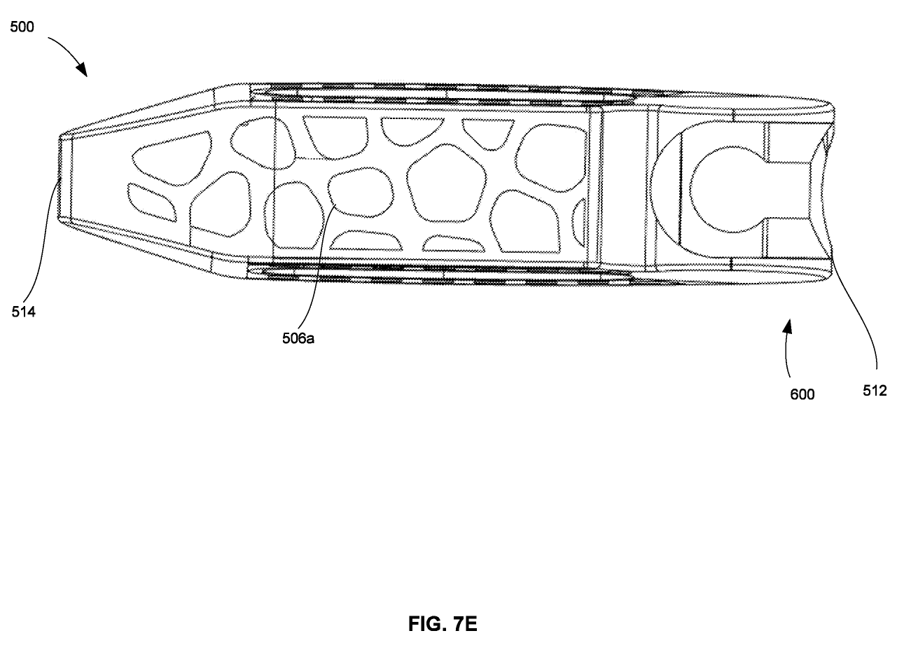

FIGS. 7A-7E depict an example implant system 500, according to an aspect of the present disclosure. Implant system 500 includes implant body 501 and inserter attachment 600.

Implant 500 includes a first lateral side 502, a second lateral side (not shown), a superior side 506a, an inferior side (not shown), a posterior end 512, and an anterior end 514. Implant 500 also includes protrusions 508 on first lateral side 502 and second lateral side (not shown), a window 516 through first lateral side 502 and second lateral side (not shown), and a recess 510 on posterior end 512.

The outer configuration of implant 500 has a generally curved, kidney bean-like shape, and thus is consistent with the shapes of interbody implants used in TLIF techniques. However, the shape of implant 500 is not the most important feature, and the implant may have a different shape in other aspects. Protrusions 508 on first lateral side 502 and on second lateral side (not shown) are square. However, the protrusions on implant 500 can be any size and shape that can engage a bone. Window 516 is a substantially oval shape to receive, for example, bone graft material. However, in other aspects, implant 500 may include any number of windows of any size and shape. Additionally, in other aspects, implant 500 may not include any windows. Superior side 506a and inferior side (not shown) are porous. However, in other aspects, implant 500 may not be porous or may include a combination of porous and nonporous sections.

Implant body 501 includes a recess 510 at the posterior end. Recess 510 is sized and shaped so to receive inserter attachment 600. As shown in FIG. 7B, inserter attachment 600 is spherical and includes a longitudinal slit 604 defining a first portion 602a and a second portion 602b. Inserter attachment has a width larger than a width of an opening of recess 510 of implant body 501 so that first and second portions 602a, 604b of inserter attachment 600 initially compress upon entry in recess 510. Once fully received, first and second portions 602a, 604b of inserter attachment 600 relax from the compressed position upon entry. As such, inserter attachment 600 is secured within recess 510.

Inserter attachment 600 has an internally threaded portion 518 so as to receive a threaded inserter to insert implant body 501 into a patient. However, in other aspects, the inserter attachment may include any mechanism to connect to an inserter.

Implant body 501 may be manufactured through additive manufacturing, and inserter attachment 600 may be manufactured through subtractive manufacturing.

A method of using implant system 500 includes assembling implant body 501 and inserter attachment 600 by inserting inserter attachment 600 into recess 510 of implant body 501. Upon entry, first and second portions 602a, 604b of inserter attachment 600 initially compress. Once fully received, first and second portions 602a, 604b relax from the compressed position so that inserter attachment 600 is secured within recess 510.

Once received in recess 510, inserter attachment 600 is rotated around axis B-B 90 degrees until a tool (not shown) is removed from inserter attachment 600. The tool (not shown) may be connected to inserter attachment 600 by a tangible connection, thereby enabling the tool to be removed from the inserter attachment 600.

The inserter attachment 600 remains in recess 510 to attach an inserter (not shown) to inserter attachment 600. For example, an inserter may be threadedly attached to inserter attachment 600. Attaching an inserter (not shown) to inserter attachment 600 enables functional rotation with the inserter (not shown). Implant system 500 with an inserter attached may then be inserted into a patient.

Implant 100 and inserter 200, implant 300 and inserter 400, and implant 500 and inserter attachment 600 each allow users to transition from one functional state to another with minimal user manipulation because the point of rotation is within an implant’s geometry. Thus, the designs provide an improved user experience by eliminating difficulty assembling an implant to an inserter, enabling a more time-efficient method of manipulating an orientation of an implant through the one-handed passive and continuous rotation mechanism, without sacrificing connection security or range. Additionally, a user has increased freedom of control. A smaller turning radius prevents extraneous material bulk at a distal end of the inserter, therefore preventing impeding a user’s view of the implant and exacerbating pressure on surrounding soft tissues.

Though the present disclosure is discussed in connection to TLIF spinal implants, the post may be implemented in other types of medical implants, including, but not limited to, a posterior lumbar interbody fusion (PLIF) spinal implant.

The implants of the present disclosure, implants 100, 300, or 500, may be made of any material suitable for implanting into a human body, including, but not limited to, any one of or any combination of titanium and its alloys, stainless steel and its alloys, magnesium and its alloys, cobalt and its alloys including a cobalt chrome alloy, nickel and its alloys, silver, tantalum, and niobium, or biocompatible polymers, such as but not limited to any one of or any combination of polyethylene (PE) and variations thereof, polyetheretherketone (PEEK), polyetherketone (PEK), acrylonitrile butadiene styrene (ABS), silicone, and cross-linked polymers.

Implant 100, 300, 500 may be formed layer-by-layer using an additive layer manufacturing (ALM), i.e., 3D printing, process so no separate connection mechanism is necessary to bring together any of the components of such implants. In some aspects, ALM processes are powder-bed based and involve one or more of selective laser sintering (SLS), selective laser melting (SLM), and electron beam melting (EBM), as disclosed in U.S. Pat. Nos. 9,456,901, 7,537,664, 8,728,387, and 9,180,010 and U.S. Patent Publication No. 2006/0147332, each of which is hereby incorporated by reference in their entireties herein. Other methods of ALM, which can be used to form the herein described implants, include stereolithography (SLA), fused deposition modeling (FDM), and continuous liquid interface production (CLIP). In other aspects, implant 100, 300, 500 may be manufactured through any other known method, such as through molding.

In other aspects, post 108 of implant 100 may be welded or otherwise fixedly attached to the implant. Alternatively, the post may be removably connected to the implant, thereby permitting selective use of the post with a given implant.

Implant 100, 300, 500 may include visual indications to ensure proper implant orientation when engaged in an inserter. In some aspects, the visual indications on implants 100, 300, 500 may include laser markings.

The methods disclosed herein do not have to be performed in the precise order described. Rather, various operations can be handled in a different order or simultaneously, and operations may be added or omitted.

The disclosure set forth herein includes any possible combinations of the particular features set forth above, whether specifically disclosed herein or not. For example, where a particular feature is disclosed in the context of a particular aspect, arrangement, configuration, or arrangement, that feature can also be used, to the extent possible, in combination with and/or in the context of other particular aspects, arrangements, configurations, and arrangements of the technology, and in the technology generally.

Furthermore, although the technology herein has been described with reference to particular features, it is to be understood that these features are merely illustrative of the principles and applications of the present technology. It is therefore to be understood that numerous modifications, including changes in the sizes of the various features described herein, may be made to the illustrative arrangements and that other arrangements may be devised without departing from the spirit and scope of the present technology. In this regard, the present technology encompasses numerous additional features in addition to those specific features set forth in the claims below. Moreover, the foregoing disclosure should be taken by way of illustration rather than by way of limitation as the present technology is defined by the claims set forth below.

1. An inserter for a spinal implant comprising:
an inner shaft including a longitudinal slit at a distal end defining a first arm and a second arm, each arm including a concave portion on an internal surface, the concave portions defining a space; and
an outer shaft surrounding a portion of the inner shaft and comprising a grasping pin positioned within the space between the first and second arms, the grasping pin having a diameter larger than a first distance between the first and second arms at a proximal end of the space so that, when the grasping pin is positioned at the proximal end of the space, the first and second arms are in an expanded position.

2. The inserter of claim 1, wherein the diameter of the grasping pin is larger than a second distance between the first and second arms at a point between the proximal end and a distal end of the space so that, when the grasping pin is positioned at the point between the proximal and distal ends of the space, the first and second arms are in a neutral position.

3. The inserter of claim 1, wherein the outer shaft surrounds a portion of the first and second arms when the grasping pin is positioned at a distal end of the space so that the first and second arms are compressed.

4. The inserter of claim 1, wherein the space is a teardrop shape so that a distance between the first and second arms at the proximal end of the space is smaller than a distance between the first and second arms at a distal end of the space.

5. The inserter of claim 1, wherein the first arm and second arm each include a rounded end, the rounded ends facing each other and defining an engagement space configured to receive a portion of an implant.

6. The inserter of claim 1, further comprising a lever pivotally connected to the inner shaft such that depression or elevation of the lever relative to the outer shaft translates the inner shaft in a proximal-distal direction.

7. The inserter of claim 6, further comprising a lever lock slidable relative to the inner shaft.

8. The inserter of claim 7, wherein the lever further comprises a tab configured to be secured under the lever lock.

9. The inserter of claim 1, further comprising a slap hammer attachment at a proximal end of the inserter, the slap hammer attachment comprising internal threading.

10. An inserter for a spinal implant comprising:
a handle including an opening at a proximal end;
an inner shaft translatable within the handle, the inner shaft comprising a grasper assembly at a distal end and a fin at a proximal portion; and
an unlocking button through the opening of the handle and surrounding the inner shaft, the unlocking button including a barrier disposed within a hole through which the inner shaft is received, the barrier abutting a distal end of the fin.

11. The inserter of claim 10, wherein a diameter of the hole is larger than the fin so that, when the unlocking button is compressed, the fin is received through the hole.

12. The inserter of claim 10, wherein the fin includes a ramped surface that tapers to a proximal end.

13. The inserter of claim 10, wherein the unlocking button comprises a top portion transverse to a side portion, the top portion including a spring configured to control movement of the unlocking button along an axis when the unlocking button is compressed.

14. The inserter of claim 10, wherein the grasper assembly includes a first arm and a second arm, the first arm and second arm each including a rounded end, the rounded ends facing each other and defining an engagement space configured to receive a portion of an implant.

15. A method of inserting a spinal implant comprising:
translating inner shaft along an axis in a distal direction so that a grasping pin connected to an outer shaft surrounding a portion of the inner shaft is moved to a proximal end of a space within a grasper assembly, wherein the grasper assembly is at a distal end of the inner shaft, and wherein the grasping pin has a width larger than a distance at the proximal end of the space and perpendicular to the axis such that a first arm and a second arm in the grasper assembly are moved to an expanded position;
engaging a post within a recess of an implant between a grasper assembly; and
translating the inner shaft in a proximal direction so that an outer shaft surrounds a portion of the inner shaft and compresses the first and second arms.

16. The method of claim 15, further comprising rotating the engaged implant about the post.

17. The method of claim 15, wherein the first translating step further comprises moving a lever pivotally connected to the inner shaft in an upward direction.

18. The method of claim 17, wherein the second translating step further comprises moving the lever in a downward direction.

19. The method of claim 17, further comprising translating a lever lock in a distal direction so that a tab on the lever is secured under the lever lock.

20. The method of claim 15, wherein the second translating step positions a proximal end of the implant against a distal end of the outer shaft, the end of the outer shaft being complementary in shape to the proximal end of the implant.
